(19) United States
(12) Patent Application Publication
(10) Pub. No.: US 2011/0130425 A1
(43) Pub. Date: Jun. 2, 2011

(54) Use of H3 Histaminergic Agonists for the Treatment of Addiction to Drugs of Abuse

(76) Inventors: Jordi Ortiz de Pablo, Barcelona (ES); David William Self, Dallas, TX (US)

(21) Appl. No.: 12/953,733

(22) Filed: Nov. 24, 2010

(30) Foreign Application Priority Data
Nov. 26, 2009 (ES) P200931072

(51) Int. Cl. A61K 31/4164; A61K 31/4172; A61K 31/454; A61K 31/417; A61P 25/32; A61P 25/36

(52) U.S. Cl. 514/326; 514/400

(57) ABSTRACT

The present invention relates to H₃ histaminergic agonists, compositions thereof, and methods for treating or preventing addiction to drugs of abuse in a subject. The methods comprise administering to a subject in need thereof an effective amount of one or more H₃ histaminergic agonists to prevent addiction, diminish the voluntary seeking of drugs of abuse, facilitate the cessation of the addictive behavior, and to prevent relapse.

### CROSS-REFERENCE TO RELATED APPLICATIONS

This application claims priority under 35 U.S.C. §119 to Spanish Patent Application No. P200931072, filed Nov. 26, 2009, the entire contents of which are incorporated herein by reference.

### BACKGROUND OF THE INVENTION

1. Field of the Invention

The present invention relates in general to the use of H₃ histaminergic agonists and their precursors for the prevention/treatment of substance addiction, and more particularly to mitigate craving and to prevent relapse.

2. Background Art

Substance addiction, such as drug abuse, and the resulting addiction-related behavior are enormous social and economic problems that continue to grow with devastating consequences. Addiction, physical and/or psychological dependence, is caused by a combination of genetic, drug-induced, and environmental factors, and is often considered a chronic relapsing disease. Substance addiction can occur by use of legal and illegal substances. Cocaine, nicotine, alcohol, amphetamine, methamphetamine, heroin, morphine, and other addictive substances are readily available and routinely used by large segments of the United States population (See, e.g., Office of National Drug Control Policy. The Economic Costs of Drug Abuse in the United States: 1992-2002. Washington, D.C.: Executive Office of the President (Publication No. 207303), 2004; Centers for Disease Control and Prevention. Annual Smoking—Attributable Mortality, Years of Potential Life Lost, and Productivity Losses—United States, 1997-2001. Morbidity and Mortality Weekly Report 54(25):625-628, Jul. 1, 2005; Harwood, 2000).

It is generally known that even after prolonged periods of drug abstinence, vulnerability to relapse for prior addicts is high. Relapse can be triggered by re-exposure to the drug (see, e.g., de Wit, 1996; Childress et al. 1993; Jaffe et al., 1989), by re-exposure to drug-associated stimuli (see, e.g., Childress et al., 1999; Wallace, 1989; Ehrman et al., 1992), or by exposure to stressors (see, e.g., Ahmed & Koob, 1997; Buczek et al., 1999; Erb et al., 1996).

Many drugs of abuse are naturally occurring. For example, cocaine is a naturally occurring nonamphetamine stimulant derived from the leaves of the coca plant. It is well established that cocaine blocks dopamine reuptake, acutely increasing synaptic dopamine concentrations. However, in the presence of cocaine, synaptic dopamine is metabolized and excreted. The synaptic loss of dopamine places demands on the body for increased dopamine synthesis, as evidenced by the increase in tyrosine hydroxylase levels after cocaine administration. When the precursor supplies are exhausted, a dopamine deficiency develops. There is no approved treatment to relieve cocaine addiction. Thus, there is an need for treatment strategies that effectively relieve a patient's craving for cocaine and help to prevent relapse.

Nicotine is another frequently abused drug. The alkaloid (−)-nicotine is present in cigarettes and other tobacco products that are smoked or chewed. It has been found that nicotine contributes to various diseases, including cancer, heart disease, and respiratory disease. Tobacco use is also a risk factor for other conditions, particularly heart disease. Vigorous campaigns against the use of tobacco or nicotine have taken place, and it is now common knowledge that the cessation of tobacco use brings with it numerous unpleasant withdrawal symptoms, which include irritability, anxiety, restlessness, lack of concentration, lightheadedness, insomnia, tremor, increased hunger, weight gain, and an intense craving for tobacco.

A few pharmaceutical agents have been reported as useful to treat nicotine dependence, including nicotine substitution such as nicotine gum, transdermal nicotine patches, nasal sprays, nicotine inhalers, and bupropion, the first non-nicotinic treatment for smoking cessation. Unfortunately, nicotine substitution therapy involves the administration of nicotine itself, which frequently leads to nicotine withdrawal and subsequent relapse to use of tobacco products. Thus, there is a need for a therapy to relieve nicotine withdrawal symptoms, including the long term cravings for nicotine, having a desirable side effect profile. Varenicline (Chantix® in the USA, Champix® in Canada, Europe and other countries) is a prescription medication used to treat smoking addiction. Varenicline is a nicotinic receptor partial agonist. As a partial agonist, it both reduces cravings for and decreases the pleasurable effects of cigarettes and other tobacco products, and through these mechanisms it can assist some patients to quit smoking. Varenicline is a partial agonist of the α₄β₂ subtype of the nicotinic acetylcholine receptor. Acting as an agonist varenicline binds to, and partially stimulates, the receptor without creating a full nicotine effect on the release of dopamine. Varenicline also competitively binds to α₄β₂ receptors blocking the ability of nicotine to stimulate the central nervous mesolimbic dopamine system.

Ethanol is probably the most frequently used and abused depressant in most cultures and a major cause of morbidity and mortality. Repeated intake of large amounts of ethanol can affect nearly every organ system in the body, particularly the gastrointestinal tract, cardiovascular system, and the central and peripheral nervous systems. Gastrointestinal effects include gastritis, stomach ulcers, duodenal ulcers, liver cirrhosis, and pancreatitis. Further, there is an increased rate of cancer of the esophagus, stomach and other parts of the gastrointestinal tract. Cardiovascular effects include hypertension, cardiomyopathy and other myopathies, significantly elevated levels of triglycerides and low-density lipoprotein cholesterol. These cardiovascular effects contribute to a marked increase risk of heart disease.

Symptoms associated with ethanol cessation or withdrawal include nausea, vomiting, gastritis, and peripheral edema. Acamprosate (Campral®), and naltrexone (Revia®, Depade®, Vivitrol®, Relistor®) can be used to treat alcohol dependence (for a review, see, e.g., Spanagel, 2009). Acamprosate is a weak NMDA-receptor antagonist. The mechanism of action of naltrexone is not fully understood, but as an opioid-receptor antagonist it is generally believed to be due to the modulation of the dopaminergic mesolimbic pathway. The generally accepted treatment of ethanol addiction and withdrawal is administering a tranquilizer such a chlordiazepoxide. Disulfuram may also be administered to help in maintaining abstinence. If ethanol is consumed while on disulfuram, acetaldehyde accumulates producing nausea and hypotension. Therefore, there is a need for a therapy to relieve ethanol addiction and withdrawal symptoms having a more desirable side effect profile.

Dopamine plays a major role in addiction. Nearly all addictive drugs (including both legal and illegal substances) and even addictive activities such as compulsive eating or gambling, directly or indirectly target the brain's reward system by flooding the circuit with dopamine during reward expectancy, causing reward-seeking behavior. It has been shown that the H₃ histaminergic receptor, a presynaptic autoreceptor located both in the central and the peripheral nervous system, presynaptically inhibits the release of dopamine and a number of other neurotransmitters (i.e., it acts as an inhibitory heteroreceptor) including GABA, acetylcholine, noradrenaline, and serotonin. Depressants such as alcohol, barbiturates, and benzodiazepines work by increasing the affinity of the GABA receptor for its ligand; GABA. Narcotics such as morphine and heroin work by mimicking endorphins—chemicals produced naturally by the body which have effects similar to dopamine—or by disabling the neurons that normally inhibit the release of dopamine in the reward system. Stimulants such as amphetamines, nicotine, and cocaine increase dopamine signaling in the reward system either by directly stimulating its release, or by blocking its absorption.

Histamine H₃ receptor antagonists (e.g., A-349,821, ABT-239, clobenpropit, ciproxifan, betahistine, burimamide, conessine, impentamine, iodophenpropit, or VUF-5681, thioperamide) would therefore be expected to increase the release of H₃ receptor-regulated neurotransmitters in the brain. Histamine H₃ receptor agonists (e.g., (R)-α-methylhistamine, cipralisant, immepip, imetit, immethridine, methimepip, or proxyfan), on the contrary, lead to an inhibition of the biosynthesis and release of histamine and also of other neurotransmitters such as serotonin and acetylcholine.

Inverse agonism or selective antagonism of the histamine H₃ receptor raises brain levels of histamine and other monoamines, and inhibits activities such as food consumption while minimizing non-specific peripheral consequences. H₃ histaminergic antagonists also potentiate activities such as the repeated consumption of drugs of abuse. The application of H₃ agonists or antagonists to treat substance abuse has been limited to early stages in the addiction process during which the subject is self-administering the drug.

It is also known that the administration of H₃ histaminergic agonists or antagonists can increase or decrease the amount of drug consumed by the subject. Rat models of alcohol and Methamphetamine self-administration suggest that H₃ antagonists may potentiate voluntary drug intake. The H₃ antagonists thioperamide and clobenpropit potentiate the reinforcing effects of methamphetamine (Munzar et al., 2004). H₃ antagonists can also change self-administration of a high dose of ethanol (Lintunen et al., 2001), and potentiate cocaine self-administration (Hyytia et al., 2003).

On the other hand, there is no generalized consensus as to the effects that the administration of H₃ histaminergic agonists, such as imetit, may have on drug self-administration (see, e.g., Brabant et al., 2010)

Whether H₃ histaminergic agonists can be useful in later phases of addiction (i.e., after the self-administration phase) such as the extinction or relapse phases, remains to be investigated.

### BRIEF SUMMARY OF THE INVENTION

The present invention is directed to methods for treating addiction-related behaviors in a subject. These methods comprise administering a therapeutically effective amount of a H₃ histaminergic agonist or a precursor of a H₃ histaminergic agonist to the subject. In this respect, the present invention provides a method for pharmacologically changing addiction-related behavior of a subject suffering from addiction comprising administering to the subject a therapeutically effective amount of an H₃ histaminergic agonist, wherein said effective amount of an H₃ histaminergic agonist is sufficient to diminish, inhibit or eliminate the addiction-related behavior.

In another embodiment, the invention provides a method for pharmacologically changing addiction-related behavior of a subject suffering from addiction to drugs of abuse, the method comprising the administration of a therapeutically effective amount of an H₃ histaminergic agonist to the subject, wherein the drug of abuse causes an increase in dopaminergic transmission, and wherein the effective amount of an H₃ histaminergic agonist is sufficient to diminish, inhibit or eliminate the addiction-related behavior.

In some embodiments, the drug of abuse directly causes an increase in dopaminergic transmission in the nucleus accumbens area of the human brain. In other embodiments, the drugs of abuse indirectly cause an increase in dopaminergic transmission in the nucleus accumbems area of the human brain. In some cases, a drug of abuse can affect simultaneous regions in the nucleus accumbens and in other areas, such as the ventral tegmental area, the amygdala, or the cortex (e.g., the prefrontal cortex, the anterior cingulate cortex, or the orbitofrontal cortex). In some embodiments, the drugs of abuse activate or inhibit neuronal transmission in the ventral tegmental area of the human midbrain.

In one embodiment, the addiction-related behavior is craving. In another embodiment, the addiction-related behavior is relapse.

The invention also provides a method for treating or ameliorating the symptoms of addiction to drugs of abuse comprising administering to a subject in need thereof a therapeutically effective amount of an H₃ histaminergic agonist wherein said effective amount promotes extinction. In another embodiment, the present invention provides a method for treating or ameliorating the symptoms of addiction to drugs of abuse comprising administering to a subject in need thereof a therapeutically effective amount of an H₃ histaminergic agonist wherein said effective amount inhibits relapse. In a further embodiment, the present invention provides a method for treating or ameliorating the symptoms of addiction to drugs of abuse comprising administering to a subject in need thereof a therapeutically effective amount of an H₃ histaminergic agonist wherein said effective amount inhibits self-administration.

In one embodiment, the H₃ histaminergic agonist is selected from the group consisting of imetit, immepip, and methyl histamine. In another embodiment, the H₃ histaminergic agonist is an agonist precursor. In yet another the agonist precursor is L-histidine. The agonist precursor can also be a prodrug.

The drug of abuse can be selected from the group consisting of opiates, hallucinogens, inhalants, phencyclidine, amphetamines, cocaine, cannabis, nicotine, and alcohol. In some embodiments, the drug of abuse can be cocaine, ethanol, or nicotine. In some embodiments, the drug of abuse is a combination of drugs.

In some embodiments, the method of treatment further comprises administering an additional therapeutic agent. In one embodiment, the additional therapeutic agent is administered concurrently. In another embodiment, the additional therapeutic agent is administered sequentially.

### BRIEF DESCRIPTIONS OF THE DRAWINGS

FIG. 1 shows cocaine craving during the start of the extinction phase, during which the drug ceases to be available to the animals after having been kept in the self-administration stage for at least six weeks. The X axis shows the time in minutes and the Y axis shows the number of times that the animal presses the lever per 20 minutes period.

FIG. 2A shows cocaine craving after a single 10 mg/kg intravenous dose of the H₃ histaminergic agonist imetit (upside-down triangles). The squares correspond to the group of control rats and the normal triangles are those with dose of 3 mg/kg of imetit. The X axis shows the time in minutes and the Y axis shows the number of times that the animal presses the lever per 20 minutes period. Data are mean±SEM of 10 animals per group. The significant test refers to the comparison between untreated controls and animals treated with imetit, performed using the ANOVA and Bonferroni test (*p<0.05) (Shaffer, 1995).

FIG. 2B shows the increase in latency at the start of the extinction phase after the administration of a single intravenous 10 mg/kg dose of imetit. The left column corresponds to the control group, the middle column corresponds to the group treated with a single 10 mg/kg (i.v.) dose of imetit, and the right column corresponds to the group treated with a single 3 mg/kg (i.v.) of imetit. Data are mean±SEM of 10 animals per group. *p<0.01 vs. control, performed using the ANOVA and Dunnett's test (Zolman, 1993).

FIG. 3 shows changes in pressure on the lever after resumption of the self-administration phase. The X axis shows the time in minutes and the Y axis shows the number of times that the animal presses the lever per 20 minutes period.

FIG. 4A shows changes in pressure on the level after administration of a single 10 mg/kg dose of imetit (squares). Data are mean±SEM of 10 animals per group. *p<0.05 vs. control, analyzed by means of the statistical ANOVA and Bonferroni tests.

FIG. 4B shows changes in latency after the administration of a single 10 mg/kg (i.v.) dose of imetit (right column). Data are mean±SEM of 9 animals per group. *p<0.01 vs. control, analyzed using the statistical paired t-tests (Zolman, 1993).

FIG. 5 shows a dose/response curve in control animals during the reinforcement (self-administration) phase. The graph represents the mean±SEM. The Y axis represents the number of self-injections that the animals perform in a period of 2 hours. The X axis represents the reinforcement dose obtained each time they tap the lever, expressed in mg/kg of cocaine injected.

FIG. 6 shows a dose/response curve after the administration of a 3 mg/kg (s.c.) dose of thioperamide, an H₃ histaminergic antagonist. The blank circles represent the control animals, pretreated with saline solution (n=11) and the black squares represent the animals pretreated with thioperamide (n=10) (3 mg/kg). The graph represents the mean±SEM. The Y axis represents the number of self-injections that the animals perform in a period of 2 hours. The X axis represents the reinforcement dose obtained each time they tap the lever, expressed in mg/kg of cocaine injected.

FIG. 7 shows how rats treated with imetit, an H₃ histaminergic agonist, self-administer less cocaine when the reminder doses are very low, 0.03 mg/kg. The blank circles represent the control animals (n=11) pretreated with saline solution. The inverted black triangles represent the animals (n=12) pretreated with imetit (3 mg/kg), and the black diamonds represent the animals (n=10) pretreated with L-histidine (500 mg/kg). The graph represents the mean±SEM. The Y axis represents the number of self-injections that the animals perform in a period of 2 hours. The X axis represents the reinforcement dose obtained each time they tap the lever, expressed in mg/kg of cocaine injected.

FIG. 8 shows relapse mediated by the administering of a single dose of cocaine after the extinction phase. Three cocaine dosage levels were used to induce relapse: 0.0 mg/kg (saline solution), 0.5 mg/kg of cocaine and 2.0 mg/kg of cocaine. The number of times that the rats pressed the lever (Y axis) increased proportionately to the dose of cocaine administered (X axis) The pressure on the lever shows the rat's need to find the drug. Even so, the pressing of the lever was not rewarded with more cocaine, which differentiates this stage from that corresponding to self-administration. The graphs show the values of the mean±SEM.

FIG. 9 shows the decrease in relapse induced by the administering of a single dose of cocaine after the extinction phase, where the animals were pretreated with imetit (black bar) or L-histidine (striped bar) one hour before being injected with the cocaine reminder dose. The Y axis shows the number of times the lever was tapped to obtain the cocaine and the X axis shows the dose of cocaine obtained in the reinforcement stage. The white bars represent the control. The blank bars indicate the pretreatment with saline, the black bars indicate the pretreatment with 3 mg/kg of imetit and the striped bars indicate the pretreatment with 500 mg/kg of L-histidine. When the animals were pretreated with imetit (n=9) or L-histidine (n=11), the number of times that the lever was pressed (Y axis) diminished significantly as compared to the number of times the lever was pressed by the control animals (white bars) pretreated with saline solution (n=13). The graph represents the mean±SEM of each group studied. *p<0.05 vs. saline, analyzed by means of the statistical test of Mann-Whitney U.

FIG. 10 shows how the pretreatment with thioperamide (stippled bars) in the rehabilitation phase, prior to inducing relapse by the administering of a single dose of cocaine, does not produce a significant decline in the pressure on the lever, that is, does not prevent relapse into seeking of the drug that had been initially provoked by a single dose of cocaine. The blank bars indicate the pretreatment with saline, the stippled bars indicate the pretreatment with 3 mg/kg of thioperamide and the bars with diamonds indicate the pretreatment with 6 mg/kg of thioperamide. The graph represents the mean±SEM of each group studied. The Y axis shows the number of times the lever was tapped to obtain the cocaine and the X axis shows the dose of cocaine obtained in the reinforcement stage.

FIG. 11 is a diagram of a model summarizing the convergence of the mechanisms of action of drugs of abuse on a common circuitry in the brain's limbic system. The mesolimbic dopamine pathway includes dopaminergic neurons in the ventral tegmental area (VTA) of the midbrain and their targets in the limbic forebrain, specially the nucleus acumbens (Nac). Regardless of the distinct mechanism of action of each drug of abuse, each drug converges on the VTA and Nac with common acute functional effects.

### DETAILED DESCRIPTION OF THE INVENTION

The present invention provides methods for the treatment of addiction to drugs of abuse with H₃ histaminergic agonists. In one embodiment, the drug of abuse is cocaine. In another embodiment, the drug of abuse is alcohol (ethanol). In yet another embodiment, the drug of abuse is nicotine. More particularly, the invention is directed at mitigating the anxiety or irrepressible desire, “craving”, for the consumption of drugs and the prevention of “relapse.”

### Common Actions of Drugs of Abuse on the Mesolimbic Dopamine Pathway

The present invention provides methods of pharmacological intervention at the mesolimbic dopamine pathway, a neurochemical convergence point common to all drugs of abuse and to other non-drug-dependent addictions. Despite binding to distinct initial protein targets in the brain and periphery and eliciting distinct combinations of behavioral and physiological effects upon acute administration, all drugs of abuse cause certain common effects after both acute and chronic exposure. All drugs of abuse are acutely rewarding, which promotes repeated drug intake and leads eventually, in vulnerable individuals, to addiction. All drugs also produce similar negative emotional symptoms upon drug withdrawal, a prolonged period of sensitization, and associative learning toward drug-related environmental cues. These adaptations are thought to contribute to the intense drug craving and relapse that can persist even after long periods of abstinence (Nestler, 2005).

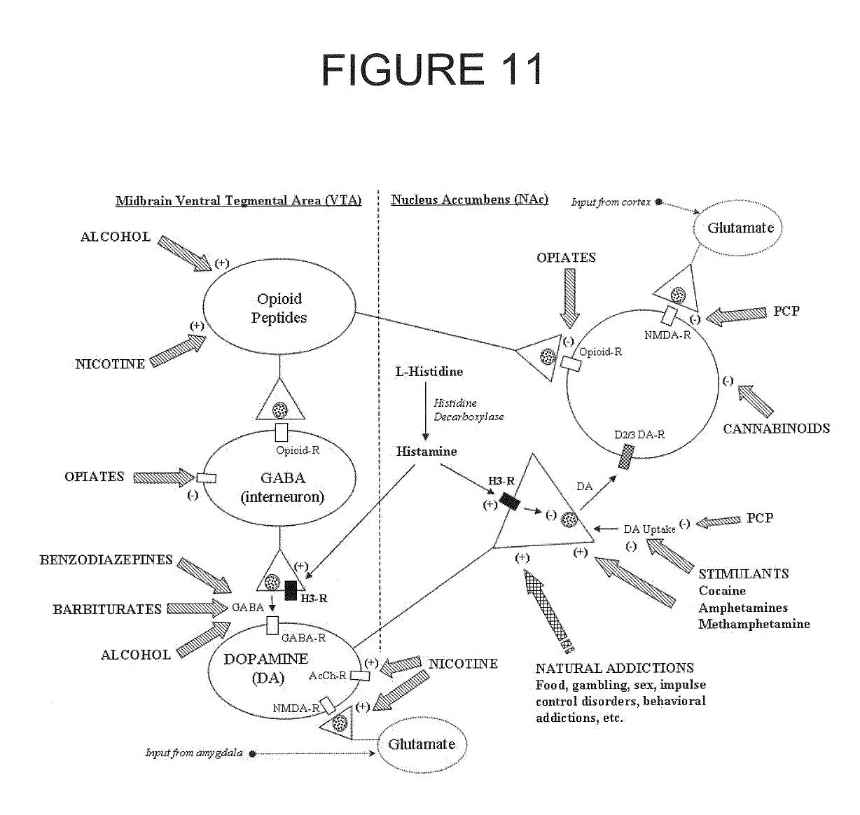

There is considerable evidence from human and animal models that all drugs of abuse converge on a common circuitry in the brain's limbic system (Koob & Le Moal, 2001; Nestler, 2001; Di Chiara et al., 2004; Volkow et al., 2004; Wise, 2004). The mesolimbic dopamine pathway includes dopaminergic neurons in the ventral tegmental area (VTA) of the midbrain and their targets in the limbic forebrain, in particular the nucleus accumbens (NAc). Regardless of the distinct mechanism of action of each drug of abuse, each drug converges on the VTA and NAc with common acute functional effects (Koob & Le Moal, 2001; Nestler, 2001; Di Chiara et al., 2004; Volkow et al., 2004; Wise, 2004; Dani et al., 2001; Boehm et al., 2004; Howlett et al., 2004) (FIG. 11).

Stimulants directly increase dopaminergic transmission in the NAc. Opiates do the same indirectly: they inhibit GABAergic interneurons in the VTA, which disinhibits VTA dopamine neurons. Nicotine seems to activate VTA dopamine neurons directly via stimulation of nicotinic cholinergic receptors on those neurons and indirectly via stimulation of its receptors on glutamatergic nerve terminals that innervate the dopamine cells. Cannabinoid mechanisms involve activation of CB1 receptors on glutamatergic and GABAergic nerve terminals in the NAc and on NAc neurons themselves. Phencyclidine (PCP) may act by inhibiting postsynaptic NMDA glutamate receptors in the NAc and dopamine reuptake. Chronic drug abuse exposure seems to sensitize the dopamine system (Everitt & Wolf, 2002; Robinson & Berridge, 2003; Kalivas, 2004). This sensitization can last long after drug-taking ceases and may relate to drug craving and relapse.

Numerous types of drugs of abuse, including cocaine, amphetamine, opiates, alcohol or nicotine, induce a long-term potentiation-like state in VTA dopamine neurons (Saal et al., 2003; Borgland et al. 2004; Thomas & Malenka, 2003; Kauer, 2004), and chronic administration of drugs of abuse, including cocaine, amphetamine, opiates, alcohol and nicotine, also increases levels of tyrosine hydroxylase (TH), the rate-limiting enzyme in dopamine biosynthesis in the VTA (Nestler, 1992; Lu et al., 2003).

These common mechanisms contribute to all drug of abuse addictions, and at least to some aspects of natural addictions (i.e., compulsive consumption of natural rewards such as food (Johnson & Kenny, 2010; Avena et al., 2008) or sex (Driver-Dunckley et al., 2007), and impulse control disorders or behavioral addictions such as gambling or shopping (Tamming a & Nestler, 2006), internet sex addiction (Bostwick & Bucci, 2008) or excessive internet game play (Han et al., 2007; see also Kelley & Berridge, 2002; Tobler et al., 2005; Grant et al., 2010; Bossong et al., 2009; Dagher et al., 2009; Voon & Fox, 2007; Voon et al., 2007; Weintraub et al., 2006).

Thus, in one embodiment, the present invention provides methods and compositions to treat addictions to drugs of abuse. In another embodiment, the present invention provides methods and compositions to treat dopamine-dependent addictions not caused by the intake of drugs of abuse.

### DEFINITIONS

To facilitate the understanding of this invention, a number of terms are defined below:

The term “agonist” refers to a compound that mimics the action of a natural neurotransmitter or causes changes at the receptor complex. The term “agonist” is used in a broad sense, and includes the natural neurotransmitter. Thus, for example, in this case, histamine is considered an H₃ receptor agonist. Histamine is derived from the decarboxylation of the amino acid L-histidine, a reaction catalyzed by the enzyme L-histidine decarboxylase. Prodrugs or compounds that are agonist precursors (e.g., L-histidine) also fall within the definition of agonist. “Prodrug” and “precursor” have been considered equivalent terms for the purpose of the present invention.

The chemical reaction by which L-Histidine is converted to histamine is shown below:

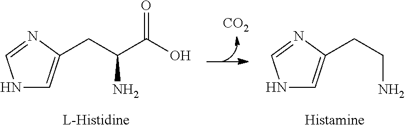

The term “antagonist” refers to a compound that binds to a receptor site, but does not cause any physiological changes.

The term “prodrug” refers to any compound metabolized in vivo to provide the bioactive agent at therapeutic doses. Prodrugs are therapeutic agents, inactive per se but transformed into one or more active metabolites. Thus, in the methods of treatment of the present invention, the terms “administer” and “treat” shall encompass treating the patient with the compound specifically disclosed, or with a compound not specifically disclosed, but that converts to the specified compound in vivo after administration to the subject.

The term “drug of abuse,” for purposes of this invention, is defined as any substance (legal or illegal) that is consumed by a mammal and as a result of said consumption, the mammal experiences addiction-related behavior, cravings for the substance, rewarding/incentive effects, and dependency characteristics, or any combination thereof.

The term “drugs of abuse” also includes combinations of drugs. For example, the mammal may be addicted to ethanol and cocaine, in which case the present invention is particularly suited for diminishing, inhibiting or eliminating the addiction-related behavior of the mammal. “Combinations of drugs” of abuse, as defined herein, include any combination of two or more drugs of abuse. Combinations of abused drugs include, for example, combinations of psychostimulants, narcotic analgesics, alcohols and addictive alkaloids as discussed above. For example, combinations of abused drugs include cocaine, nicotine, methamphetamine, ethanol, morphine and heroin. A highly abused combination of drugs is a mixture of cocaine and heroin.

As used herein “addiction-related behavior” means behavior resulting from compulsive substance use and it is characterized by apparent total dependency on the substance. Symptomatic of the behavior is (i) overwhelming involvement with the use of the drug, (ii) the securing of its supply, and (iii) a high probability of relapse after withdrawal. The active search for drugs, craving, and relapse are examples of addiction-related behavior. Compulsive and repetitive actions resulting from, for example, natural addictions and behavioral addictions also fall within the definition of “addiction-related behavior.”

Several phases or stages can be distinguished when studying addiction-related behavior:

I. Chronic Self-Administration or Acquisition Phase: During this phase the subject can obtain the drug freely and addiction to the drug develops. The drug can be self-administered by the individual, or, e.g., in the case of a patient under pain management at a hospital or a sick child, the drug may be administered by others.

II. Extinction Phase: In this phase, the drug ceases to be available. During the extinction phase, the drug can no longer be obtained, which triggers drug seeking and an irrepressible desire for the drug (craving).

III. Relapse Phase: In this phase, the desire for the drug is again induced by a new administration of the drug or by environmental factor such as stress or drug-related cues or memories.

As used herein, “craving” an abused drug or a combination of abused drugs is an intense desire to self-administer the drug(s) previously used by the mammal.

As used herein, “extinction” is defined as the process in which the frequency of the learned response to the conditioned stimulus decreases and ultimately disappears, due to the lack of reinforcement. During the extinction phase, the drug ceases to be available to the test animal, initially increasing its anxiety to obtain it. In drug addiction research, the extinction of drug-seeking behavior in animals is considered analogous to—a model for—a human addict becoming abstinent.

The term “reinforcement” is defined as the presentation of a stimulus, usually rewarding, immediately following a specific behavior, in order to increase the frequency of that behavior. Here, the inventors have trained rats to repeatedly press levers (the behavior) by arranging for the rat to receive a positive reinforcer whenever it does so. In drug experiments, the reinforcer is often food to begin with, and then the drug under investigation. The fact that a rat presses the lever to receive a dose of cocaine is taken as evidence that it finds the cocaine reinforcing (pleasurable, motivating).

As used herein, “latency” is defined as the time elapsing until the rats press the lever for the first time to obtain the reward during experimental procedures.

The term “relapse” is defined as the process of returning to the behavior and thought patterns typical of active addiction, which had been overcome by extinction learning, and which ultimately lead again to use, returning even to the stage of addictive disease that existed prior to beginning the rehabilitation. In particular, the term “relapse” relates to the taking of the drug of abuse after an abuse-free period. The most important problem in the treatment of addictions is to prevent relapse, since the desire to relapse may manifest itself even several years after having stopped taking the drug. The prediction to relapse in the final phase of addiction remains for several years and is due to persistent cellular changes.

The term “prevent” also encompasses preventing the recurrence or relapse-prevention of a disease or condition or of symptoms associated therewith, for instance after a period of improvement.

As used herein, the term “cocaine” comprises the pure alkaloid, and preparations wherein cocaine is an active ingredient, e.g., coca leaves, coca paste, cocaine hydrochloride, “crack”, etc. As used herein, the term “alcohol” and “ethanol” are interchangeable. As used herein, the term “tobacco” includes, but is not limited to, smoking materials (e.g., cigarettes, cigars, pipe tobacco), snuff, chewing tobacco, and nicotine-containing products (e.g., gum, lozenges).

The term “H₃ receptor” or “H₃ histaminergic receptor” means any of the isoforms of the histamine H₃ receptor that inhibits the release of a number of monoamines, including histamine (see, e.g., Hill et al., 1997).

The term “patient” refers to a subject suffering from addiction to drugs of abuse or at risk of suffering from addiction to drugs of abuse who has been specifically chosen to receive a therapeutic treatment. The term “subject” as used herein, refers to an animal, such as a mammal, for example, a human, who has been selected for treatment, observation or experimentation. The term “mammal” also includes animals of economic importance such as bovine, ovine, and porcine animals, especially those that produce meat, as well as domestic animals, sports animals, pets, zoo animals, and humans.

The term “treatment” as used herein refers to any treatment of a mammalian condition or disease, and includes: (1) inhibiting the disease or condition, i.e., arresting its development, (2) relieving the disease or condition, i.e., causing the condition to regress or diminishing the symptoms, or (3) stopping the symptoms of the disease. The term “inhibit” includes its generally accepted meaning which includes eliminating, prohibiting, preventing, restraining, alleviating, ameliorating, slowing, stopping, or reversing the progression or severity of a resultant symptom. As such, the present method includes prophylactic, diagnostic and therapeutic regimens.

The “effective amount” or “therapeutically effective amount” as used herein is that amount effective to achieve the specified result of changing addiction-related behavior of the mammal. It is an amount which diminishes or relieves one or more symptoms or conditions resulting from cessation or withdrawal of the psychostimulant, narcotic analgesic, alcohol, nicotine or combinations thereof, such as craving or relapse. It should be emphasized, however, that the method of the invention is not limited to any particular dose.

The term “composition” as used herein encompasses a product comprising specified ingredients in predetermined amounts or proportions, as well as any product that results, directly or indirectly, from combining specified ingredients in specified amounts. In relation to pharmaceutical compositions, this term encompasses a product comprising one or more active ingredients, and an optional carrier comprising inert ingredients, as well as any product that results, directly or indirectly, from combination, complexation or aggregation of any two or more of the ingredients, or from dissociation of one or more of the ingredients, or from other types of reactions or interactions of one or more of the ingredients.

In general, pharmaceutical compositions are prepared by uniformly and intimately bringing the active ingredient into association with a liquid carrier or a finely divided solid carrier or both, and then, if necessary, shaping the product into the desired formulation. The pharmaceutical composition includes enough of the active object compound to produce the desired effect upon the progress or condition of diseases. Accordingly, the pharmaceutical compositions of the present invention encompass any composition made by admixing a compound of the present invention and a pharmaceutically acceptable carrier. By “pharmaceutically acceptable” it is meant the carrier, diluent or excipient must be compatible with the other ingredients of the formulation and not deleterious to the recipient thereof. For the purpose of present invention the terms “pharmaceutical composition” and “medicament” must be interpreted as synonyms.

As used in the present disclosure and claims, the singular forms “a,” “an,” and “the” include plural forms unless the context clearly dictates otherwise.

It is understood that wherever embodiments are described herein with the language “comprising,” otherwise analogous embodiments described in terms of “consisting of” and/or “consisting essentially of” are also provided.

The term “and/or” as used in a phrase such as “A and/or B” herein is intended to include both “A and B,” “A or B,” “A,” and “B.” Likewise, the term “and/or” as used in a phrase such as “A, B, and/or C” is intended to encompass each of the following embodiments: A, B, and C; A, B, or C; A or C; A or B; B or C; A and C; A and B; B and C; A (alone); B (alone); and C (alone).

Throughout this application, the term “about” is used to indicate that a value includes the inherent variation of error for the device, the method being employed to determine the value, or the variation that exists among the study subjects.

Units, prefixes, and symbols are denoted in their Système International de Unites (SI) accepted form. Numeric ranges are inclusive of the numbers defining the range. The headings provided herein are not limitations of the various aspects or embodiments of the invention, which can be had by reference to the specification as a whole.

### Treatment or Prevention of Addiction-Related Behavior

As stated above, the present invention comprises methods for the treatment of behaviors related to the addiction to drugs of abuse in a subject, the methods comprising administering to the subject a therapeutically effective amount of an H₃ histaminergic agonist or a precursor of an H₃ histaminergic agonist. More precisely, the invention comprises methods directed to mitigating the anxiety or irrepressible desire (“craving”), for the consumption of drugs of abuse, and methods to prevent “relapse” into abuse.

Within the concept of drugs of abuse one can include any type of substance that produces dependency and that is used voluntarily to bring about certain sensations or mental states not justified therapeutically.

Drugs of abuse include, but are not limited, to psychostimulants, narcotic analgesics, alcohols and addictive alkaloids such as nicotine, cannabinoids, psychedelics or hallucinogens, aryl cyclohexylamines, inhalants, or combinations thereof. Drugs of abuse also include CNS depressants such as barbiturates, chlordiazepoxide, and psychoactive cannabinoids such as tetrahydrocannabinol.

Some examples of psychostimulants include but are not limited to amphetamine, dextroamphetamine, methamphetamine, phenmetrazine, diethylpropion, methylphenidate, cocaine, phencyclidine, methylenedioxymethamphetamine and pharmaceutically acceptable salts thereof.

Specific examples of narcotic analgesics include alfentanyl, alphaprodine, anileridine, bezitramide, codeine, dihydrocodeine, diphenoxylate, ethylmorphine, fentanyl, heroin, hydrocodone, hydromorphone, isomethadone, levomethorphan, levorphanol, metazocine, methadone, metopon, morphine, opium extracts, opium fluid extracts, powdered opium, granulated opium, raw opium, tincture of opium, oxycodone, oxymorphone, pethidine, phenazocine, piminodine, racemethorphan, racemorphan, thebaine and pharmaceutically acceptable salts thereof.

In one particular embodiment of the present invention, the drug of abuse is selected from the group consisting of cocaine, amphetamines, alcohol, and nicotine.

It has been demonstrated that H₃ receptor agonists (e.g., imetit) and precursors of those agonists (e.g., L-histidine) have effects in stages subsequent to self-administration. The methods of treatment disclosed here demonstrate that H₃ receptor agonists are effective during the extinction stage, when the drug is no longer available, by diminishing the tendency of the test animal to search for the drug. Furthermore, the methods of treatment disclosed demonstrate that H₃ receptor agonists prevent relapse in the taking of drugs of abuse, particularly cocaine. Thus, the present invention comprises the use of H₃ histaminergic agonists for the development of pharmaceutical compositions to prevent the addiction to drugs of abuse. The present invention is also directed to the use of H₃ histaminergic agonists for the development of pharmaceutical compositions to treat behaviors associated with the addiction to drugs of abuse, such as the voluntary search for drugs of abuse and relapse into drug abuse.

In one embodiment, the H₃ histaminergic agonists useful in the methods and compositions of the present invention include, but are not limited to imetit, immepip, impentamine, FUB 407, (R)α-methylhistamine, cipralisant, immethridine, methimepip, and proxyfan. In another embodiment, H₃ histaminergic agonists useful in the methods and compositions of the present invention include, but are not limited to precursors (e.g., L-histidine, precursor of histamine) or prodrugs (e.g., BP 2.94, a prodrug of (R)α-methylhistamine).

In one embodiment, the H₃ histaminergic agonists useful in the methods and compositions of the present invention include histamine precursors such as carnosine (see, e.g., Wu et al., 2006). In another embodiment, the H₃ histaminergic agonists useful in the methods and compositions of the present invention include drugs causing an increase in the histamine levels in the brain such as metoprine.

In one embodiment, at least one H₃ agonist or H₃ agonist precursor is administered alone to a subject in need thereof. In another embodiment, more than one H₃ agonist, H₃ agonist precursor, or combinations thereof are administered. In one embodiment, several H₃ agonists, H₃ agonist precursors, or combinations are administered concurrently. In some embodiments, the H₃ agonists, H₃ agonist precursors, or combinations thereof are administered sequentially. In some embodiments, H₃ agonists, H₃ agonist precursors, or combinations of H₃ agonists and H₃ agonist precursors are administered in combination with one or more antagonists. In one embodiment, the H₃ agonists, H₃ agonist precursors, or combinations thereof are administered concurrently with one or more antagonists. In another embodiment, the H₃ agonists, H₃ agonist precursors, or combinations thereof are administered sequentially with one or more antagonists. In one embodiment, H₃ agonists, H₃ agonist precursors, or combinations thereof are administered as part of a course of treatment comprising other pharmacological treatments of behaviors associated with the addiction to drugs of abuse known in the art, such as varenicline, acamprosate, naltrexone, bupropion, or disulfiram.

In yet another embodiment, treatments with combinations of H₃ agonists, H₃ agonist precursors, H₃ antagonists, and/or other drugs are alternated with the administration of H₃ agonists, H₃ agonist precursors, or antagonists alone. The administration of H₃ agonists, H₃ agonist precursors, antagonists, and/or other drugs can alternate with the administration of H₃ agonists, H₃ agonist precursors, or antagonists alone according to one of many patterns, such as ACACA, CACAC, ACCCA, CCACC, CCCCA, ACCCC, etc.

The H₃ agonist can be administered to a subject prior, during, or after the subject has become addicted to a drug of abuse. In one embodiment, the H₃ agonist is administered before the administration of the drug of abuse has commenced as a preventive agent. In another embodiment, the H₃ agonist is administered while the subject is self-administering or receiving the drug of abuse. In yet another embodiment of the present invention, the H₃ agonist is administered as an adjuvant during the extinct phase. In one embodiment, the H₃ agonist is administered to prevent craving during the reinforcement phase. In another embodiment, the H₃ agonist is administered to prevent or delay the onset of relapse.

The H₃ agonists are administered at a concentration that is therapeutically effective. Clinical applications may be derived from the studies disclosed in the examples since doses used are within clinical standards and since administration at the doses herein reported appears to be safe. The United States Department of Agriculture and the National Cancer Institute have indicated that extrapolation of animal doses to humans can be correctly performed through normalization to body surface area (see, e.g., Reagan-Shaw et al., 2008). Thus, the Human Equivalent Dose (HED) can be calculated by the following formula: HED (mg/kg)=Animal Dose (mg/kg)×(Animal Kₘ/Human Kₘ), using Kₘ factors of 3, 6, and 37 for mice, rats and humans, respectively. Consequently, a HED of a certain compound is 8.1% and 16.2% of the dose administered to a mouse or rat, respectively.

In some embodiments, the range of H₃ histaminergic agonist doses administered to a subject in need thereof is from about 50 mg/kg to about 200 mg/kg. The agonist dose can be, for example, about 50 mg/kg, about 75 mg/kg, about 100 mg/kg, about 125 mg/kg, about 150 mg/kg, about 175 mg/kg, or about 200 mg/kg. The dose range for a H₃ histaminergic agonist of the invention is the range in which the administration of the agonist is safe and effective, i.e., the range in which the amount of agonist or a composition comprising the agonist is high enough to significantly positively modify the condition to be treated, but low enough to avoid serious side effects (at a reasonable benefit/risk ratio).

The amount of H₃ agonist to be administered is readily determined by one of ordinary skill in the art without undue experimentation. Factors influencing the mode of administration and the respective amount of H₃ agonist include, but are not limited to, the severity of the addiction to the drug of abuse, the history of addiction of the subject, and the age, height, weight, health, and physical condition of the subject. Similarly, the amount of H₃ agonist to be administered is dependent upon the mode of administration and whether the subject receives a single dose or multiple doses of the H₃ agonist. Generally, a higher dose of H₃ agonist is desired with increased weight of the subject undergoing treatment. Thus, in some conditions it may be necessary to use doses outside the ranges stated above.

As one of skill in the art understands, other factors influence the ideal dose regimen in a particular case. Such factors can include, for example, the binding affinity of the H₃ agonist and the half-life of the H₃ in the bloodstream, the desired steady-state H₃ agonist concentration level, frequency of treatment, and the influence of other therapies used in combination with the H₃ agonist and the treatment method of the invention.

While it may be possible for the compounds disclosed to be administered as the raw chemical, these compounds are typically administered as a “pharmaceutical composition.” According to a further aspect, the present invention provides a pharmaceutical composition comprising at least one H₃ agonist or H₃ agonist precursor, at least one pharmaceutically acceptable salt or solvate thereof, or a mixture of any of the foregoing, together with one or more pharmaceutically acceptable carriers thereof, and optionally one or more other therapeutic ingredients. The carrier(s) must be “acceptable” in the sense of being compatible with the other ingredients of the formulation and not deleterious to the recipient thereof. The H₃ agonist or H₃ agonist precursor or drug combination comprising a H₃ agonist or H₃ agonist precursor can be formulated using a variety of carriers, adjuvants, diluents, excipients, or any combinations thereof known in the art.

As explained above, single or multiple administrations of H₃ agonists and compositions comprising H₃ agonist can be administered depending on the dosage and frequency as required and tolerated by the patient. In any event, the composition should provide a sufficient quantity of the H₃ agonist of this invention to effectively treat the patient. Generally, the dose should be sufficient to treat or ameliorate cravings or to prevent or reduce the likelihood of relapse without producing unacceptable toxicity to the subject. An effective amount of the H₃ agonist or composition comprising the H₃ agonist is that which provides either subjective relief of a symptom or symptoms or an objectively identifiable improvement as noted by the clinician or other qualified observer.

In some embodiments, a single treatment course is sufficient to achieve the expected behavioral response. In other embodiments, multiple rounds of treatment are required, Typically, the frequency of dosing depends upon the pharmacokinetic parameters of the H₃ agonist in the formulation used. Generally, the H₃ agonist is administered until a dosage is reached that achieves the desired effect. The H₃ agonist can therefore be administered as a single dose or multiple doses, at the same time or difference concentration/dosages, or as a continuous infusion. Further refinement of the dosage is routinely made. Appropriate dosages can be ascertained through the use of the appropriate dose-response data. It will also be appreciated that the effective dosage of H₃ agonist used for treatment or prevention can increase or decrease over the course of a particular treatment. Changes in dosage can result and become apparent from the results of diagnostic methods prior, during, or after the therapy.

In some embodiments of the invention, the subject is treated with escalating doses of the H₃ agonist. Typically, the subject receives an initial dose of the H₃ agonist, and the dose is escalated until the desired effect is achieved or the toxicity becomes unacceptable.

In other embodiments of the present invention, the H₃ agonist is administered in a dosage, such that an effective amount is provided to the subject based on biological properties, such as pharmacokinetic parameters or combinations thereof. Examples of those pharmacokinetic parameters include arithmetic peak plasma concentration (Cₘₐₓ), biological half-life (T_{1/2}), arithmetic area under the curve from time zero to infinity (AUC_{0∞}, and clearance rate (Cl). For instance, the formulation administered through the methods of the invention can be selected such that when administered to a subject in need thereof, the selected formulation provides the subject with one or more of the desired pharmacokinetic parameters.

Any means for delivering the agonist or its precursor may be employed. For example, the agonist may be administered orally, intravenously, subcutaneously, sublingually, or by means of a transdermal patch. The H₃ agonists can be administered by injection, for example, by intravenous infusion (i.v.). Intravenous administration can occur by intravenous infusion over a time period. The infusion can be given over longer or shorter time periods as required.

Bioavailability of an H₃ agonist or agonist precursor such as L-histidine may be enhanced by mechanical dissolution enhancement by embedding the active ingredient in a matrix of suitable inert material to be ingested or injected. Bioavailability may also be enhanced by chemically modifying the agonist or adding molecules, substituents, or protective groups which protect the agonist or agonist precursor against metabolization or enzymatic degradation. Bioavailability may also be enhanced by the manufacture of a prodrug whereby an ingredient is attached or linked to the agonist or agonist precursor which allows the agonist or agonist precursor to escape first-pass metabolism and which enhances bioavailability. The production of a prodrug can also be used to overcome problems associated with stability, toxicity, lack of specificity. Bioavailability may also be enhanced by a transdermal skin-patch like formulation which would bypass the first pass metabolism and achieve direct penetration into the blood and brain.

It is contemplated that any embodiment discussed in this specification can be implemented with respect to any method, kit, reagent, or composition of the invention, and vice versa. Furthermore, compositions of the invention can be used to achieve methods of the invention. It will be understood that particular embodiments described herein are shown by way of illustration and not as limitations of the invention.

The following examples are set forth to assist in understanding the invention and should not, of course, be construed as specifically limiting the invention described and claimed herein. Such variations of the invention, including the substitution of all equivalents now known or later developed, which would be within the purview of one skilled in the art and changes in formulation or minor changes in experimental design, are to be considered to fall within the scope of the invention incorporated herein.

### Example 1

### General Methodology Used in Animal Studies

The characteristic symptoms of addiction to drug abuse in humans have been evaluated in laboratory rat models. The H₃ histaminergic agonists used in the present invention were tested in said rats with the goal of studying whether their administration was able to revert the addictive stage of said experimental animals, in each of the stages of the addiction process, particularly during the stages of extinction and relapse.

The research methodology comprises various phases:

I. Chronic Self-Administration or Acquisition Phase: During this phase the rats can obtain the drug by pressing on a lever, that is, the drug is absolutely available to the rats. The quantity of drug which the rats desire over the course of time is measured during this phase.

II. Extinction Phase: In this phase, the drug ceases to be available to the rats. During the extinction session, the animal learns, suddenly, that the drug can no longer be obtained by pressing the lever, which directs the rat toward an initial impulse of drug seeking. During the extinction session, one estimates the degree of the irrepressible desire of the rat for the drug (craving). After repeated extinction sessions, animals learn to reduce lever pressing and are ready for relapse testing.

III. Relapse Phase: The relapse phase is studied by using a rehabilitation test. In this phase, the desire of the rats for the drug is again induced by the administration of a single dose of drug. At this instant, the rat again feels the desire to obtain the drug, and so it again begins to press on the lever to obtain the drug, but the drug is not available to the animal.

### Example 2

### Self-Administration Phase

### Selection of Experimental Animals and Induction of Addiction

Training was conducted in Skinner cages. Rats were trained to tap a lever to obtain saccharose. During this training rats were gradually deprived of food, which resulted in a 10% decrease in body weight. Once the training to obtain saccharose was complete, the rats had access to food ad libitum during the rest of the procedure. An intravenous catheter was subsequently implanted in the jugular vein. The catheter passed underneath the skin to reach the shoulder of the animal, where it was secured with a surgical gauze.

After 3-4 days of recovery, the animals were subjected, in the Skinner cages, to daily sessions of intravenous self-administration of 0.5 mg/kg doses of cocaine each time that they pressed an active lever. The Skinner cages also contained an inactive level. When this second lever was pressed by the animal, no drug was administered. The self-administration sessions lasted for 2 hours a day, 5 days a week, for at least six weeks.

After this first training step in the self-administration phase, the animals that (a) were capable of discriminating between the active and inactive levers, and (b) exhibited stability in the taking of the drug (i.e., 10% maximum variability for 3 consecutive days), were subjected to a second self-administration protocol in which they were required to press the lever three times to obtain the cocaine reinforcement.

Rats that had learned the first and second self-administrations protocols and exhibited stability in the triple pressing of the lever to obtain the drug, were subjected to a third learning protocol. The third self-administration protocol required the animals to press the lever five times to obtain the cocaine reinforcement.

Rats that succeeded in learning to self-administer the drug according to all three protocols described above were selected to test out the efficiency of the treatment with H₃ histaminergic agonists in the extinction and relapse phases.

### Example 3

### Extinction Phase

### Evaluation of the Effect of the H₃ Histaminergic Agonists

Drug craving, in this particular case cocaine craving, associated with the unexpected commencement of the extinction phase was studied once the animals had been self-administering the drug daily for at least six weeks.

The experiments began with the self-administration of cocaine for one hour. After this initial self-administration, the rats were divided into two groups. One group was administered a saline solution (control group) and the second group was administered the H₃ histaminergic agonist imetit intravenously, at two different doses (3 mg/kg or 10 mg/kg).

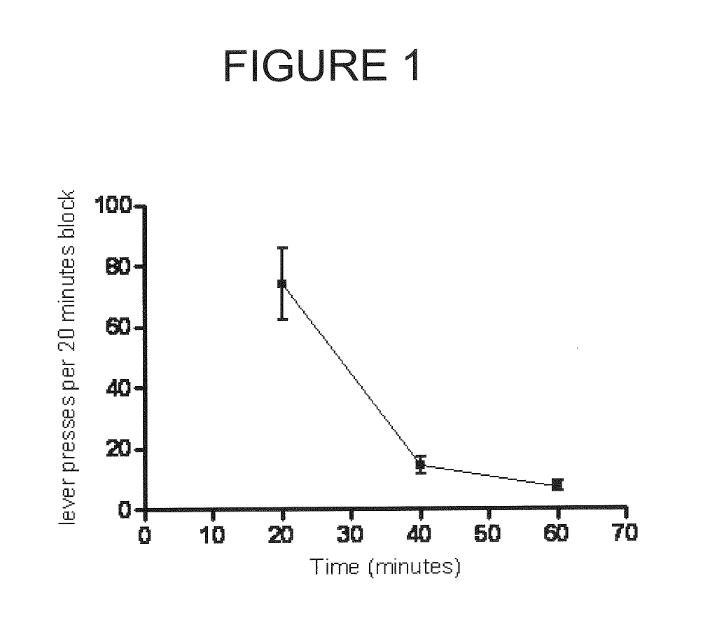

The rats were then returned to the rooms under conditions of extinction, i.e., the drug was not available to them, even if they pressed the lever that had previously been provided to them. In the control group, an increase in the impulsive pressing of the lever was observed during the first 20 minutes of extinction. This impulse diminished over time (FIG. 1)

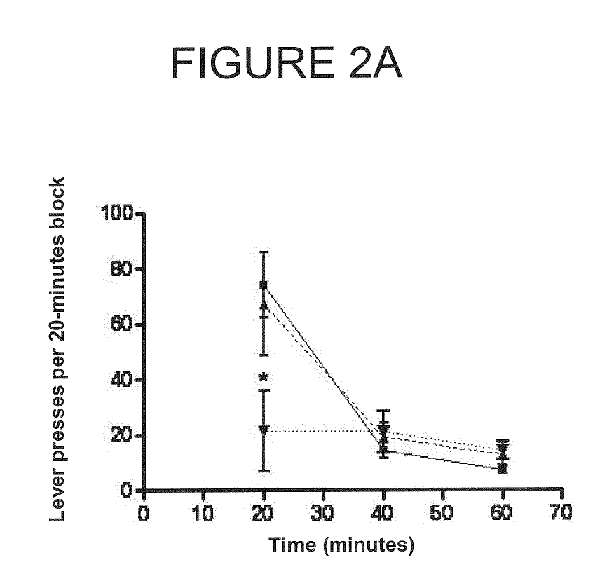

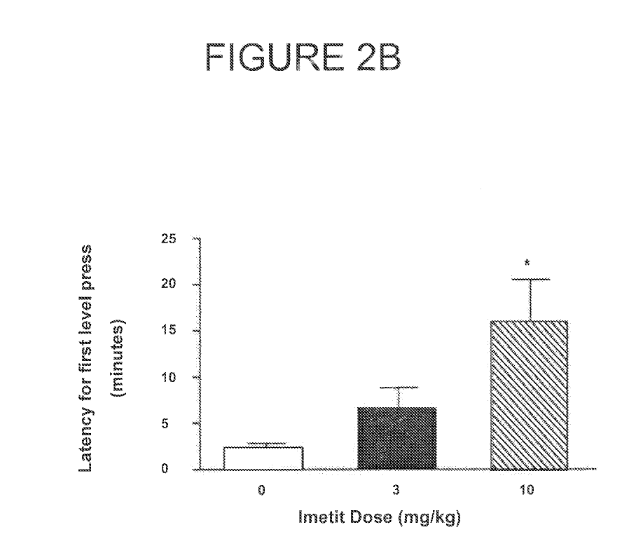

In the groups of rats treated with imetit the treatment with the high dose, 10 mg/kg, significantly diminished in the first 20 minutes the impulsiveness of the animals in tapping the lever to obtain reward as compared to the group of control rats and the group of rats treated with 3 mg/kg of imetit (FIG. 2A). The number of taps by the control rats was between 60 and 85 in the first 20 minutes of extinction, and administration of low imetit doses (3 mg/kg) did not cause a significant reduction in tapping. The treatment of the animals with the high dose of imetit significantly increased latency, i.e., the time elapsing until the rats pressed the lever for the first time to obtain the reward (FIG. 2B).

After these treatments, the rats recovered stability by means of three sessions of self-administration of the drug.

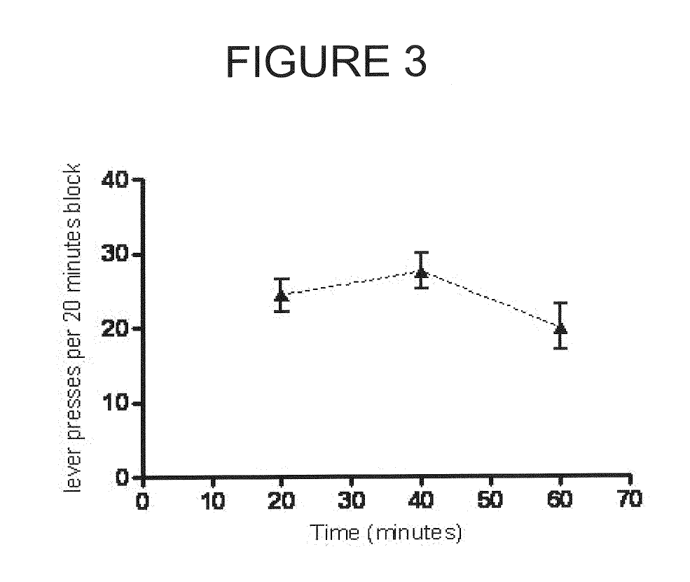

In order to differentiate the drug seeking from the reward phenomenon, the same protocol as described above was used, with the proviso that the pressing of the lever was rewarded with drug during the second hour. In the control experiments, when the rats were treated with saline solution, a relatively stable pressing of the lever over the course of time was observed (FIG. 3).

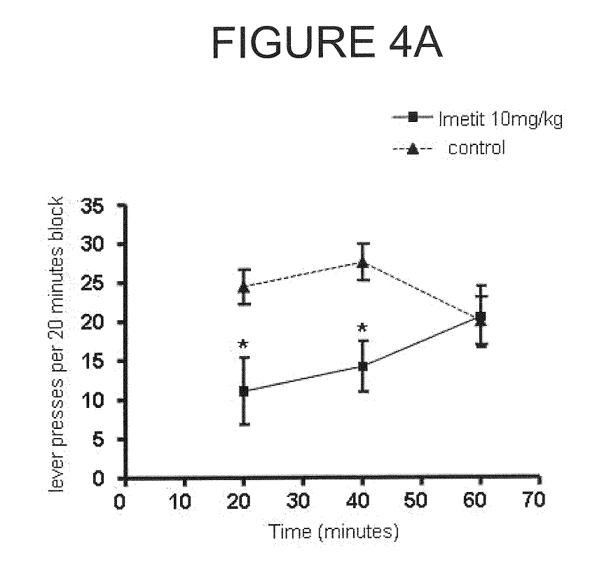

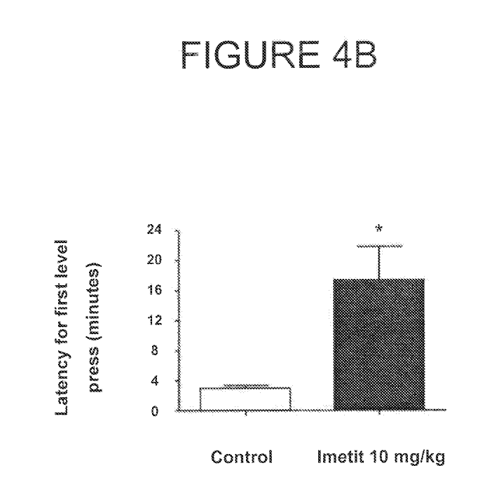

These same rats, days afterwards, were subjected in random order to treatment with saline solution or imetit in doses of 10 mg/kg. After these treatments, the rats recovered stability by means of three sessions of self-administration of drug. Pretreatment with imetit at a dose of 10 mg/kg significantly diminished, by approximately 50%, the number of times that the rats tapped the lever during the start of the self-administration phase (FIG. 4A). This decrease was significant for the first 40 minutes of the self-administration period, as compared to the control rats that were pretreated with a saline solution. Thus, over the course of time, the desire for the drug reappeared. The pretreatment of the animals with 10 mg/kg of imetit significantly increased, with respect to the control rats, the latency or time elapsing until the rats pressed the lever for the first time to obtain the reward (FIG. 4B).

### Example 4

### Reinforcement Phase

### Effect of H₃-Histaminergic Agonists on Cocaine Self-Administration

The effect of histaminergic drugs on the reward or reinforcement produced by cocaine was evaluated in animals that had self-administered various doses of cocaine. First, the animals went through a phase of daily self-administration of cocaine (0.5 mg/kg) for a minimum period of six weeks. Once the rats showed stability in this daily self-administration regime, they were randomly assigned to five different experimental groups. The rats in the control group received an injection of saline solution each time they tapped the lever. The other four groups of rats received a different reinforcement dose of cocaine (0.03 mg/kg, 0.1 mg/kg, 0.3 mg/kg and 1 mg/kg per injection, respectively) each time they tapped the lever.

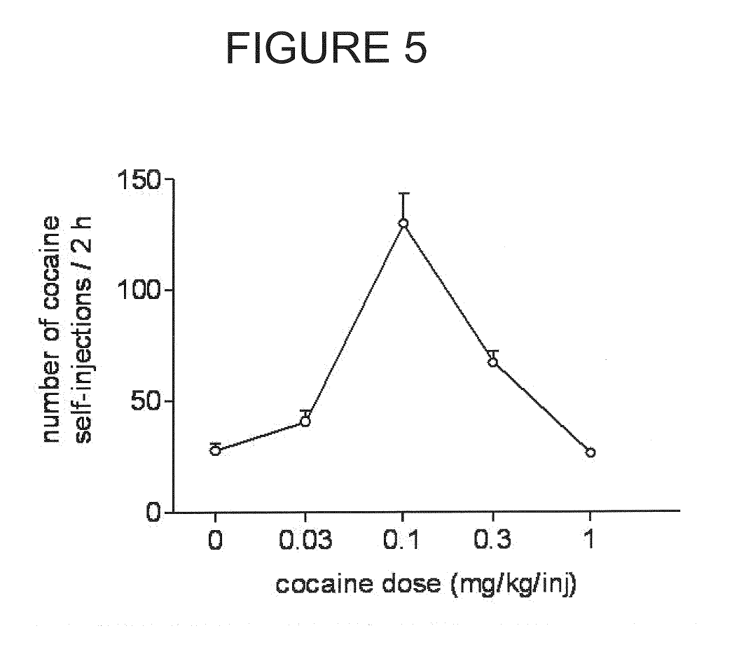

Then, the number of times that the animals pressed the lever to obtain their dose of cocaine was recorded. The number of taps recorded indicated the quantity of cocaine desired by the animal. Plotting the number of time that animals pressed the level versus the cocaine dose showed a typical inverted-U dose/response curve (FIG. 5). Low doses of cocaine did not provide a reward and the high doses of cocaine allowed the animal to take similar quantities of cocaine with fewer taps on the lever, whereas the intermediate dose of 1 mg/kg per injection was such that the animals exhibited the greatest impulsiveness in tapping the lever.

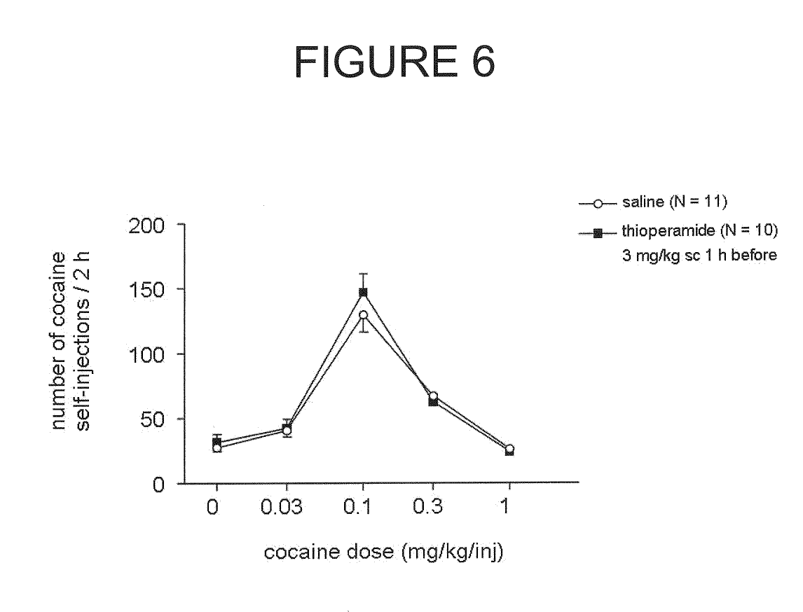

Next, the effect of histaminergic drugs on the reinforcement produced by cocaine was evaluated Rats were treated with a single 3 mg/kg dose of thioperamide, an H₃ histaminergic antagonist, one hour before being subjected to cocaine reinforcement trials at doses of 1.0 mg/kg, 0.3 mg/kg, 0.1 mg/kg, 0.03 mg/kg and 0 mg/kg per injection. The treatment with thioperamide produced no significant increase in the reinforcement produced by the different doses of cocaine that were studied. The observed inverted-U shaped dose/response curve was similar to that observed in the control animals that were not treated with thioperamide (FIG. 6).

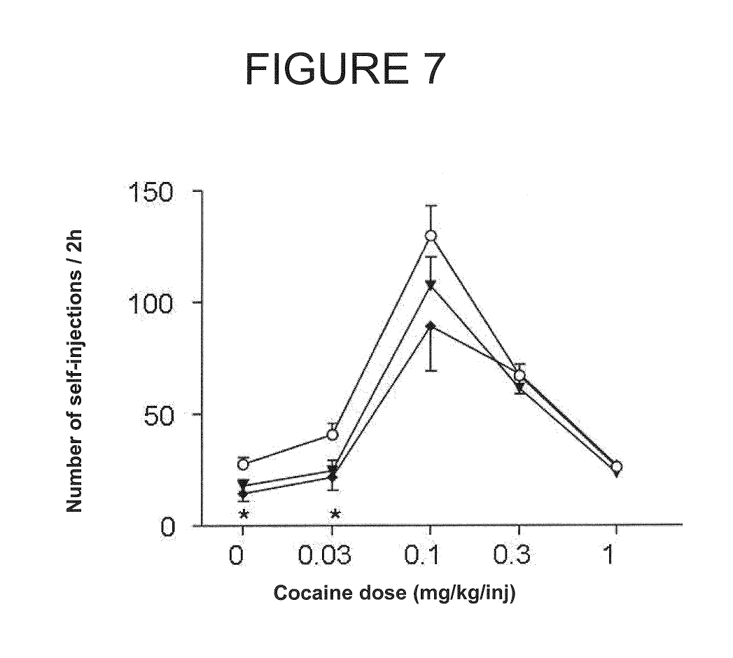

Pretreatment with a single 3 mg/kg dose of imetit, an H₃ histaminergic agonist, or a single 500 mg/kg dose of L-histidine, a histamine precursor, one hour before reinforcement diminished the tendency to seek cocaine (FIG. 7). This effect was observed even at the lowest cocaine reinforcement dose, i.e., 0.03 mg/kg, (FIG. 7).

### Example 5

### Relapse Phase

### Evaluation of the Effects of H₃ Histaminergic Agonists

The behavior of the rats was also studied during the relapse phase, after the animals had gone through the extinction phase. In the extinction phase, the animals received no reward (dose of cocaine) when they pressed the lever. After a minimum of six extinction sessions, lasting 2 hours each session, carried out on consecutive days, the lever-pressing behavior of the rats diminished at least 20 times in comparison to the start of the extinction sessions.

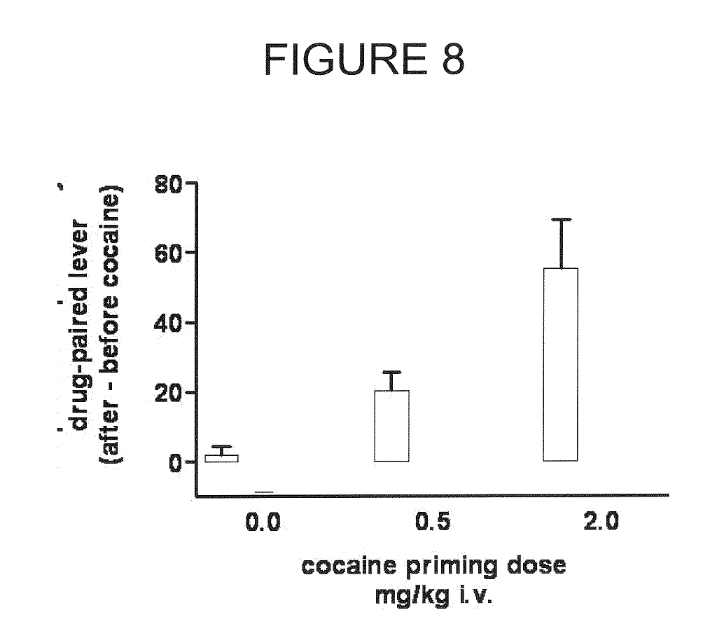

When the number of times that the rats pressed the lever decreased, the animals were subjected to 4 hour rehabilitation sessions (i.e., relapse sessions). These rehabilitation sessions consisted initially of extinction sessions of 3 hours, followed by the automatic release, by an injection, of a saline solution or different doses of cocaine. During the fourth hour, the behavior of the animals was studied in regard to the number of times that they pressed the lever after the administration of the different doses of cocaine (0 mg/kg, 0.5 mg/kg and 2 mg/kg). The number of times that the lever was pressed increased in proportion to the dose of cocaine administered (cocaine at 0.5 mg/kg or 2 mg/kg administered intravenously, or saline in controls) (FIG. 8). However, this increase in the number of times that the animals pressed the lever was not rewarded with the administration of more cocaine. The number of times that the rats pressed the lever was indicative of the rat's need to find the drug. Even so, the pressing of the lever was not rewarded with more cocaine, which differentiates this stage from that corresponding to self-administration.

To evaluate the effect of H₃ agonists and antagonists on the relapse phase, one hour before beginning the rehabilitation study, the animals were randomly divided into different experimental groups. The control group (n=13) was treated with saline solution. A group of rats (n=9) was treated with a 3 mg/kg dose of imetit (H₃ agonist). Another group of rats (n=7) was treated with a 3 mg/kg subcutaneous dose of thioperamide (H₃ antagonist). The third group (n=7) was treated with a 6 mg/kg dose of thioperamide. The last group of animals (n=11) was treated with a 500 mg/kg intraperitoneal dose of L-histidine (histamine precursor).

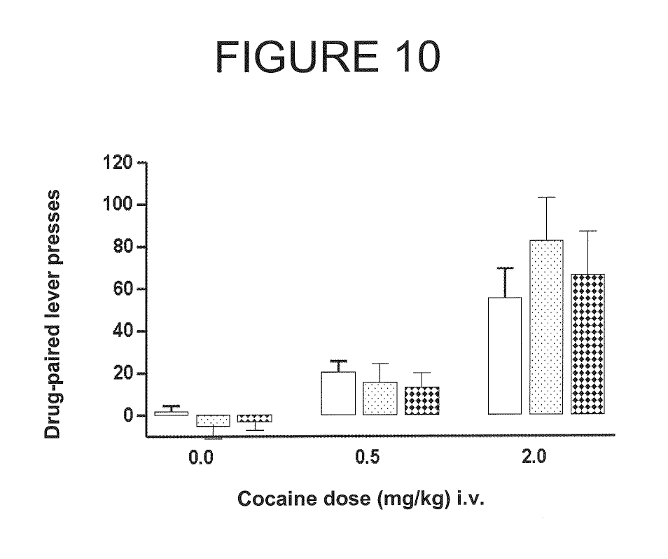

The pretreatment of the rats with the different doses of H₃ histaminergic antagonist thioperamide did not potentiate the rehabilitation of the animals in whom relapse was induced by the administration of a reminder dose of 0.5 mg/kg or 2 mg/kg (FIG. 10), i.e., the H₃ antagonist thioperamide does not diminish the desire for cocaine in the rehabilitation phase

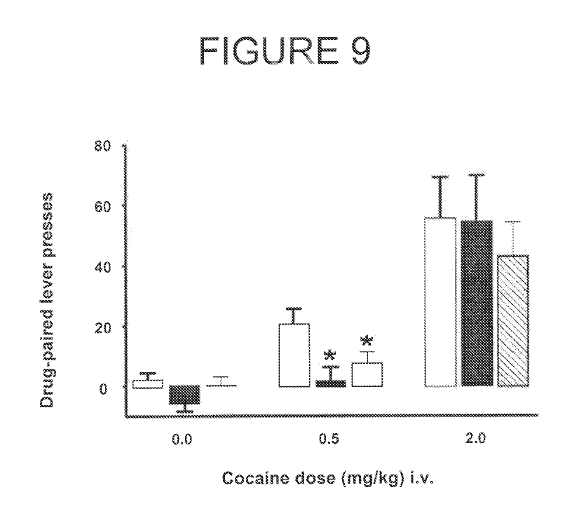

In contrast, pretreatment with imetit (H₃ agonist) or L-histidine (H₃ agonist precursor) caused a decrease in the pressing on the lever, i.e., the pretreatment prevented the relapse into drug seeking that was initially provoked by a single dose of cocaine of 0.5 mg/kg (FIG. 9).

Therefore, whereas H₃ histaminergic antagonists do not promote the rehabilitation of animals in which relapse has been induced, the administration of H₃ histaminergic agonists or their precursors promote the rehabilitation of the animals in which relapse has been induced by the administration of the drug of abuse.

In summary, the results presented above show a two-fold effect of the administration of H₃ histaminergic agonists in rats. Treatment with H₃ agonist leads to a decrease in the voluntary drug seeking at the start of the extinction phase, and also a decrease in the tendency of a subject to relapse into the taking of said drug.

### Example 6

### Effects of L-Histidine on Relapse into Seeking Alcohol Self-Administration

Fourteen male Sprague-Dawley rats obtained from Charles-River weighing 200 g at the beginning of the protocol are used. Rats are water-deprived for the few hours before the first overnight session. Rats are placed in an operant chamber (Panlab) with two levers. Pressing the “active” lever provides access to 0.1 ml of a 20% sucrose solution under a continuous reinforcement program. A light above the active lever signals availability of the solution in a drinking chamber for 20 seconds. Pressing the “inactive” lever has no consequences, although it is recorded by the software (Packin, Panlab).

The following daily sessions take 60 minutes and water is provided ad libitum. When stable patterns of sucrose self-administration are achieved, sucrose is decreased progressively until reaching 0% while ethanol is proportionally increased until it reaches 10%. Afterwards, the program is increased to Fixed Ratio 2 (FR2), i.e., two lever presses give access to one reinforcement. Self-administration is maintained next to FR3 and finally to FR5. Animals reaching criteria for (i) stable FR5 lever press for 3 consecutive days (20% variation), (ii) correctly discriminating active and inactive levers, and (iii) having consumed ethanol daily (5 days per week) for a minimum of 4 weeks are considered ready for extinction and reinstatement procedures.

Before extinction sessions, saline is administered orally (5 ml/kg) to all rats. For extinction training, rats are placed in the same operant chambers but no alcohol access or cue light is contingent on lever press. Once rats decrease lever press to less than 10 presses per hour they are considered ready for reinstatement (relapse) procedures.

Before the reinstatement sessions, saline 5 ml/kg or L-histidine 1 g/kg (dissolved in saline and adjusted to pH 5) are administered orally to the rats. Then reinstatement sessions start with a 60 minutes control extinction session after which 0.5 mg/kg alcohol (12% w/v in saline) is administered intraperitoneally. Lever press during the following 60 minutes after alcohol intraperitoneal administration are recorded, but the lever presses do not have any consequences.

Rats completing all these procedures are again allowed to self-administer alcohol daily. If these rats meet criteria for stable lever press and discrimination of active and inactive levers, they undergo newly the extinction and reinstatement procedures. However, in this case, rats previously in the control group are treated with L-histidine, and rats previously treated with L-histidine are treated as controls, in a cross-over latin square design.

Rats completing both protocols undergo for a third time the alcohol self-administration, extinction, and relapse procedures under control conditions, to monitor possible deviations of the results due to learning the procedure. Finally, rats are subject to a fourth process of self-administration and extinction, finishing with a relapse session induced by 5 minutes of intermittent foot-shock stress (0.5 mA; 0.5 seconds on; mean off period of 40 seconds) applied to the grid floor of the cage. After the foot-shock stress procedure rats are not placed again into the self-administration chambers (Lê et al., 1998; Lê et al., 2002)

The administration of L-histidine decreases the reinstatement of the alcohol seeking behavior caused by the exposure to ethanol. Also, the administration of L-histidine decreases the reinstatement of the alcohol seeking behavior caused by stress.

### Example 7

### Effects of L-Histidine on Nicotine Stimulation of Dopamine Release

One male Sprague-Dawley rat obtained from Charles-River weighing 200-250 g is used. The rat brain is rapidly removed and placed in ice-cold oxygen-saturated (O₂/CO₂:95%/5%) Krebs bicarbonate buffer (124 mM NaCl, 0.8 mM KCl, 1.25 mM NaH₂PO₄, 1.2 mM KH₂PO₄, 0.67 mM MgSO₄, 2.6 mM CaCl₂, 10 mM glucose and 27.5 mM NaHCO₃, pH 7.4). The striata is dissected and cut using a Mcllwain™ Mechanical Tissue Copper (Stoelting Co., Wood Dale, Ill.) into miniprisms of 0.3 mm/side. The microprims are transferred to ice-cold Krebs buffer, gently centrifuged, and the buffer changed to remove damaged tissue and cellular debris.

Brain miniprisms are distributed into 24 flat-bottom Eppendorf tubes and preincubated for 2 hours in 0.25 mL Krebs buffer at 37° C. using an Eppendorf Thermomixer® under a O₂/CO₂ atmosphere. Then the medium is replaced with fresh Krebs buffer at 37° C. with or without 0.5 mM L-histidine prepared in the same buffer. This concentration of L-histidine is similar to that reached in plasma of L-histidine-treated humans (Block et al., 1967).

After 20 minutes of incubation, 0.1 μM ³H-tyrosine (10000 dpm/tube; Perkin Elmer) is added to all tubes. After another 10 minutes, the slices are slightly depolarized for 5 minutes with 15 mM K⁺ by addition of concentrated KCl containing nicotine. The final concentration of nicotine in the sample, 100 nM, is comparable to nicotine concentrations reached in the brains of smokers. Concentrations ten times higher can also be used, but they cause desensitization of nicotinic receptors more easily (Pidoplichko et al., 1997).

All samples are then cooled down on ice and quickly centrifuged at 1000×g for 1 minute to separate buffer from tissue. 100 nM dopamine with 3 mM ascorbic acid and 0.5% (v/v) trichloroacetic acid is added to all samples as internal standard. Blank samples contain the same components, except that ³H-tyrosine is added after the trichloroacetic acid, and they are left on ice during the entire procedure. All samples are sonicated on ice for 15 seconds and centrifuged (13000×g, 20 minutes, 4° C.). Supernatants are injected into an automated HPLC to purify newly synthesized and released dopamine. The HPLC system uses a Tracer Extrasil ODS2 5 μm 25×0.46 cm reverse phase column (Teknokroma) with a 2×20 mm guard column. The mobile phase contains 0.1 M NaH₂PO₄, 0.75 mM octanesulfonic acid, 1 mM EDTA adjusted to pH 5, plus 12% methanol. Samples are run isocratically at 1 mL/minute. An automatic autosampler Merck-Hitachi L-7200 and an automated fraction collector Gilson FC203B automatically recover internal standard dopamine peaks when UV detected into scintillation vials.

Tritium contained in the scintillation vials represents ³H-dopamine synthesized from ³H-tyrosine in the brain miniprisms. The percentage of released dopamine in each tube is obtained by dividing ³H-dopamine in the buffer after the incubation by the total ³H-dopamine (buffer+tissue) synthesized in the same tube (Rosell et al., manuscript in preparation).

Nicotine potentiates K⁺-elicited dopamine release through stimulation of α₄β₂ nicotinic acetylcholine receptors. L-Histidine is taken up by histaminergic terminals where histamine is formed and released by K⁺ depolarization, acting on histamine receptors. As a result, slices preincubated with L-histidine show less dopamine release caused by nicotine stimulating than the controls.

### Example 8

### Effects of an H₃ Histaminergic Agonist on the Extinction and Reinstatement of Nicotine Seeking Behavior Using a Mouse Intravenous Operant Self-Administration Paradigm

Operant intravenous self-administration in animals is the most frequently used and reliable method to assess drug reinforcing effects, as well as the extinction and reinstatement of drug seeking behavior. Animals intravenously self-administer compounds almost exclusively abused by humans; however, the specific patterns of intake, extinction, and reinstatement are comparable to those observed in human subjects.

### Materials:

L-Histidine solution: L-Histidine (Sigma, H-8000) at a 155.15 g/mol concentration in 0.34 M phosphoric acid is prepared as follows. 1.5 g L-histidine are dissolved in 12 ml of 0.34 M phosphoric acid to obtain a 0.8 M solution of L-histidine at approximately pH 6. pH is verified and 0.1 g of NaCl added to a final concentration of 0.9%. Finally, the L-histidine solution is sterilized by filtration.

Solutions of other H₃ histaminergic agonists or H₃ histaminergic agonist precursors are prepared according to their physicochemical properties.

Animals: Male C57B16/J mice (Charles River, France) are housed individually in controlled laboratory conditions with the temperature maintained at 21±1° C., humidity at 55±10%, and light reversal cycle (light on at 20.00 h, light off at 08.00 h). Mice are tested during the dark phase of a light reversal cycle. Behavioural tests and animal care are conducted in accordance with the standard ethical guidelines (National Institutes of Health 1995; European Communities Directive 86/609 EEC) and the protocols are approved by the local ethical committee (CEEA-PRBB).

Apparatus: The self-administration experiments are conducted in mouse operant chambers (Model ENV-307A-CT, Medical Associates, Georgia, VT, USA). The experimental chambers are equipped with a house light, ventilator fan, drug infusion pump, liquid swivel with counterbalanced arm, and two manipulanda with cue lights that are located on either side of a food dipper. The manipulanda are holes (1.2 cm diameter). One manipulanda is selected as active hole for delivering the reinforcer and the other as inactive hole. Nose-poking on the active hole results in a reinforcer (nicotine infusion) while nose-poking on the inactive hole has no consequences. The chambers are housed in sound- and light-attenuated boxes equipped with fans to provide ventilation and white noise. A stimulus light, located above the active hole, is paired contingently with the delivery of the reinforcer.

### Methods:

Surgery for drug self-administration: Mice are anaesthetized under isoflurane anesthesia (1.5-2.0%) and then implanted with indwelling i.v. silastic catheters as previously described (Caine et al., 1999) with minor modifications. Briefly, a 6 cm length of silastic tubing (0.3 mm inner diameter, 0.6 mm outer diameter) (Silastic®, Dow Corning, Houdeng-Goegnies, Belgium) is fitted to a 22 gauge steel cannula (Semat, Herts, England) that is bent at a right angle and then embedded in a cement disk (Dentalon Plus, Heraeus Kulzer, Germany) with an underlying nylon mesh.

The catheter tubing is inserted 1.3 cm into the right jugular vein and anchored with suture. The remaining tubing runs subcutaneously to the cannula, which exits at the midscapular region. All incisions are sutured and coated with antibiotic ointment (Bactroban, GlaxoSmithKline, Spain). After surgery, animals are allowed to recover for 3 days prior to initiation of self-administration sessions.

The catheter is flushed daily with a saline solution containing heparin (30 UI/mL) in order to maintain its patency. The patency of intravenous catheters is evaluated periodically (approximately every 6 days) and whenever drug self-administration behavior appears to deviate dramatically from that observed previously. Patency is evaluated by the infusion of 0.1 ml of thiobarbital (5 mg/ml) through the catheter. If prominent signs of anesthesia are not apparent within 3 seconds of the infusion, the mouse is removed from the experiment.

L-Histidine dosage: The first L-histidine dose is 1 g/kg. The second dose of L-histidine is 2 g/kg. Dosage for other H₃ histaminergic agonists or H₃ histaminergic agonist precursors is determined considering the pharmacokinetic and toxicological characteristics of the compound.

Drug self-administration, extinction and reinstatement procedure: Nicotine self-administration sessions are performed based on protocols previously described (Soria et al., 2005). Briefly, sessions start 3 days after surgery. Responding is maintained by nicotine (75 micrograms/kg per injection) delivered in 23.5 μl over 2 seconds. Nicotine is infused via a syringe that is mounted on a microinfusion pump (PHM-100A, Med-Associates, Georgia, VT, USA) and connected via Tygon tubing (0.96 mm o.d., Portex Fine Bore Polythene Tubing, Portex Limited, Kent, England) to a single channel liquid swivel (375/25, Instech Laboratories, Plymouth Meeting, Pa., USA) and to the mouse intravenous catheters. The swivel is mounted on a counter-balanced arm above the operant chamber.

Two hours daily self-administration sessions are conducted 6 days per week. The house light is on at the beginning of the session for 3 seconds and off during the remaining time of the session.

Each daily session starts with a priming injection of the drug. First, mice are trained under a Fixed Ratio 1 (FR1) reinforcement schedule. A 30 second time-out period is established after each reinforcement, During this 30 second period, the cue light is off and no reward is provided on the active hole. Responses on the inactive hole and all the responses during the 30 second time-out period are also recorded. The session terminates after 30 reinforcers are delivered or after 2 hours, whichever occurs first. The stimulus light signals delivery of the reinforcer. Operant training on FR1 is performed during at least 10 days. The criteria for the acquisition are achieved when mice maintain a stable responding with (i) less than 25% deviation from the mean of the total number of reinforcers earned in three consecutive sessions (75% of stability), (ii) at least 75% responding on the active hole, and (iii) a minimum of 5 reinforcers per session. After each session, mice are returned to their home-cages.

Once the acquisition criteria is achieved, nicotine is substituted by saline, starting the extinction phase that lasts until responding on the active hole is lower than 40% of the mean response during the stable acquisition for two consecutive days. The compound is administered before each extinction session during the initial extinction training (10 days). Route and time of administration before the test are determined according to the pharmacokinetic characteristics of the compound.

The reinstatement of nicotine seeking behaviour induced by the conditioned environmental cue (stimulus-light) is tested in those mice achieving the criteria for extinction. Mice are exposed to a first reinstatement session after receiving vehicle or compound administration. The operant behaviour is then extinguished in subsequent extinction training sessions. Finally, mice are exposed to a second reinstatement session after receiving the counterbalanced vehicle or compound administration (see experimental groups).

Animals are distributed in three types of experimental of groups, (a) acquisition groups, (b) extinction groups, and (c) reinstatement groups.

(a) Acquisition groups: group 1 (nicotine, IV, self-administered, n=9), group 2 (nicotine, IV, self-administered, n=9), group 3 (nicotine, IV, self-administered, n=9),

(b) Extinction groups: group 1 (vehicle administration during the initial extinction training, 10 days), group 2 (compound administration (first dose) during the initial extinction training, 10 days), group 3 (compound administration (second dose) during the initial extinction training, 10 days),

(c) Reinstatement groups: group 1 (vehicle (first reinstatement test)+compound (effective dose) (second reinstatement test)), group 2 (compound (first dose) (first reinstatement test)+vehicle (second reinstatement test)), croup 3 (compound (second dose) (first reinstatement test)+vehicle (second reinstatement test)),

wherein the “compound” is, e.g., an H₃ histaminergic agonist or an H₃ histaminergic agonist precursor such as L-histidine.

The number of animals refers to the total mice reaching the self-administration acquisition criteria, although a higher number of mice is required to achieve this number in the experiment.

The effects of the administration of the compound are tested in the following main responses: (i) temporal pattern of responding during the first extinction session, (ii) duration and achievement of the extinction criteria, (iii) effects on the reinstatement of drug seeking behaviour (within group comparison), and (iv) effects on the reinstatement of drug seeking behaviour (between group comparison).

The administration of L-histidine (1) decreases or delays responding during the first extinction session, (2) shortens the number of sessions required to achieve the extinction criteria, and (3) decreases cue-induced reinstatement of nicotine seeking. These conclusions are reached by either (i) comparison between control groups and groups of L-histidine-treated mice, or (ii) repeated measures (also called within-group comparison) of the same mice subjected to two reinstatement sessions under control or L-histidine-treated conditions.

### Example 9

### Effects of an H₃ Histaminergic Agonist on the Extinction and Reinstatement of Morphine/Heroin Seeking Behaviour Using a Mouse Intravenous Operant Self-Administration Paradigm

Operant intravenous self-administration in animals is the most frequently used and reliable method to assess drug reinforcing effects, as well as the extinction and reinstatement of drug seeking behavior. Animals intravenously self-administer compounds almost exclusively abused by humans; however, the specific patterns of intake, extinction, and reinstatement are comparable to those observed in human subjects.

### Materials:

L-Histidine solution: L-Histidine (Sigma, H-8000) at a 155.15 g/mol concentration in 0.34 M phosphoric acid is prepared as follows. 1.5 g L-histidine are dissolved in 12 ml of 0.34 M phosphoric acid to obtain a 0.8 M solution of L-histidine at approximately pH 6. pH is verified, and 0.1 g of NaCl added to a final concentration of 0.9%. Finally, the L-histidine solution is sterilized by filtration.

Solutions of other H₃ histaminergic agonists or H₃ histaminergic agonist precursors are prepared according to their physicochemical properties.

Animals: Male C57B16/J mice (Charles River, France) are housed individually in controlled laboratory conditions with the temperature maintained at 21±1° C., humidity at 55±10%, and light reversal cycle (light on at 20.00 h, light off at 08.00 h). Mice are tested during the dark phase of a light reversal cycle. Behavioural tests and animal care are conducted in accordance with the standard ethical guidelines (National Institutes of Health 1995; European Communities Directive 86/609 EEC) and the protocols are approved by the local ethical committee (CEEA-PRBB).

Apparatus: The self-administration experiments are conducted in mouse operant chambers (Model ENV-307A-CT, Medical Associates, Georgia, VT, USA). The experimental chambers are equipped with a house light, ventilator fan, drug infusion pump, liquid swivel with counterbalanced arm, and two manipulanda with cue lights that are located on either side of a food dipper. The manipulanda are holes (1.2 cm diameter). One manipulanda is selected as active hole for delivering the reinforcer and the other as inactive hole. Nose-poking on the active hole results in a reinforcer (nicotine infusion) while nose-poking on the inactive hole has no consequences.

The chambers are housed in sound- and light-attenuated boxes equipped with fans to provide ventilation and white noise. A stimulus light, located above the active hole, is paired contingently with the delivery of the reinforcer.

### Methods:

Surgery for drug self-administration: Mice are anaesthetized under isoflurane anesthesia (1.5-2.0%) and then, implanted with indwelling i.v. silastic catheters as previously described (Caine et al., Psychopharmacology 147: 22, 1999) with minor modifications. Briefly, a 6 cm length of silastic tubing (0.3 mm inner diameter, 0.6 mm outer diameter) (Silastic®, Dow Corning, Houdeng-Goegnies, Belgium) is fitted to a 22 gauge steel cannula (Semat, Herts, England) that is bent at a right angle and then embedded in a cement disk (Dentalon Plus, Heraeus Kulzer, Germany) with an underlying nylon mesh.

The catheter tubing is inserted 1.3 cm into the right jugular vein and anchored with suture. The remaining tubing runs subcutaneously to the cannula, which exits at the midscapular region. All incisions are sutured and coated with antibiotic ointment (Bactroban, GlaxoSmithKline, Spain). After surgery, animals are allowed to recover for 3 days prior to initiation of self-administration sessions. The catheter is flushed daily with a saline solution containing heparin (30 UI/mL) in order to maintain its patency. The patency of intravenous catheters is evaluated periodically (approximately every 6 days). Patency is also evaluated whenever drug self-administration behavior appears to deviate dramatically from that observed previously. Patency evaluation is performed by infusing 0.1 ml of thiobarbital (5 mg/ml) through the catheter. If prominent signs of anesthesia are not apparent within 3 seconds of the infusion, the mouse is removed from the experiment.

L-Histidine dosage: The first L-histidine dose is 1 g/kg. The second dose of L-histidine is 2 g/kg. Dosage for other H₃ histaminergic agonists or H₃ histaminergic agonist precursors is determined considering the pharmacokinetic and toxicological characteristics of the compound.

Drug self-administration, extinction and reinstatement procedure: Morphine/heroin self-administration sessions are performed based on protocols previously described (Soria et al., 2005). Briefly, sessions start 3 days after surgery. Responding is maintained by morphine/heroin (0.3/0.1 mg/kg per injection) delivered in 23.5 μl over 2 seconds. Morphine/heroin is infused via a syringe that is mounted on a microinfusion pump (PHM-100A, Med-Associates, Georgia, VT, USA) and connected, via Tygon tubing (0.96 mm o.d., Portex Fine Bore Polythene Tubing, Portex Limited, Kent, England) to a single channel liquid swivel (375/25, Instech Laboratories, Plymouth Meeting, Pa., USA) and to the mouse intravenous catheters. The swivel is mounted on a counter-balanced arm above the operant chamber.

One hour daily self-administration sessions are conducted 6 days per week. The house light is on at the beginning of the session for 3 seconds and off during the remaining time of the session. Each daily session starts with a priming injection of the drug. First, mice are trained under a Fixed Ratio 1 (FR1) schedule of reinforcement. A 30 seconds time-out period is established after each reinforcement. During this 30 seconds period, the cue light is off and no reward is provided on the active hole. Responses on the inactive hole and all the responses during the 30 seconds time-out period are also recorded. The session terminates after 30 reinforcers are delivered or after 1 hour, whichever occurs first.

The stimulus light signals delivery of the reinforcer. Operant training on FR1 is performed during at least 10 days. The criteria for the acquisition is achieved when mice maintain a stable responding with less than 25% deviation from the mean of the total number of reinforcers earned in three consecutive sessions (75% of stability), with at least 75% responding on the active hole, and a minimum of 5 reinforcers per session. After each session, mice are returned to their home-cages.

Once achieved the acquisition criteria, morphine/heroin is substituted by saline. At this moment, the extinction phase starts and lasts until responding on the active hole is lower than 40% of the mean response during the stable acquisition for two consecutive days. The compound is administered before each extinction session during the initial extinction training (10 days). Route and time of administration before the test are determined considering the pharmacokinetic characteristics of the compound.

The reinstatement of morphine/heroin seeking behaviour induced by the conditioned environmental cue (stimulus-light)/non-contingently morphine/heroin priming is tested in those mice achieving the criteria for extinction. For this purpose, mice are exposed to a first reinstatement session after receiving vehicle or compound administration. The operant behavior is then extinguished in subsequent extinction training sessions. Finally, mice are exposed to a second reinstatement session after receiving the counterbalanced vehicle or compound administration (see experimental groups).

Animals are distributed in three classes of experimental of groups, (a) acquisition groups, (b) extinction groups, and (c) reinstatement groups.

(a) Acquisition groups: group 1 (morphine/heroin, IV, self-administered, n=10), group 2 (morphine/heroin, IV, self-administered, n=10), and group 3 (morphine/heroin, IV, self-administered, n=10),

(b) Extinction groups: group 1 (vehicle administration during the initial extinction training, 10 days), group 2 (compound administration (first dose) during the initial extinction training, 10 days), and group 3 (compound administration (second dose) during the initial extinction training, 10 days),

(c) Reinstatement groups: group 1 (vehicle (first reinstatement test)+compound (effective dose) (second reinstatement test)), group 2 (compound (first dose) (first reinstatement test)+vehicle (second reinstatement test)), and group 3 (compound (second dose) (first reinstatement test)+vehicle (second reinstatement test)),

wherein the “compound” is, e.g., an H₃ histaminergic agonist or an H₃ histaminergic agonist precursor such as L-histidine.

Number of animals refers to the total mice reaching the self-administration acquisition criteria, although a higher number of mice is required to achieve these criteria.

The effects of the administration of the compound is tested in at least the following main responses: (i) temporal pattern of responding during the first extinction session, (ii) duration and achievement of the extinction criteria, (iii) effects on the reinstatement of drug seeking behaviour (within group comparison), (iv) effects on the reinstatement of drug seeking behaviour (between group comparison).

The administration of L-histidine (i) decreases or delays responding during the first extinction session, (2) shortens the number of sessions required to achieve extinction criteria, and (3) decreases cue-induced reinstatement of morphine/heroine seeking.

### Example 10

### Studies on the Role of a H₃ Histaminergic Ligand on the Extinction and Reinstatement of Alcohol Seeking Behavior

### Animal Model for Relapse Behavior and Craving

A new animal model of alcoholism—long-term alcohol self-administration with repeated deprivation phases has been developed and extensively applied (Spanagel & Holier, 1999; Spanagel, 2003). This model is ideally suited to study relapse behavior, and it has been validated with acamprosate (Spanagel et al., 1996; Holter et al., 1997; Bachteler et al., 2005) and naltrexone (Witter & Spanagel, 1999; Spanagel & Holier, 2000). Such pharmacological validation demonstrates the predictive value of our animal model and enables us to further characterize putative anti-craving drugs and neurobiological mechanisms of addictive behavior. Numerous compounds have been studied using this animal model, including drugs mainly acting on opioid receptors (Hölter & Spanagel, 1999; Spanagel & Hölter, 2000; Hölter et al., 2000a), glutamate receptors (Hölter et al., 1996; Hölter et al., 2000b; Bäckström et al., 2004; Vengeliene et al., 2005; Vengeliene et al., 2007) and dopamine receptors (Vengeliene et al., 2006). An overview of these pharmacological intervention studies is provided in Spanagel & Zieglgänsberger (1997), and Spanagel & Kiefer (2008).

Another gold-standard model in the study of mechanism of addictive behavior is the reinstatement model which allows the measurement of alcohol-seeking responses. This animal model also allows the study of the motivational component of alcohol craving (Spanagel, 2003; Spanagel & Kiefer, 2008). This model has been extensively used in our laboratory to test numerous putative anti-craving compounds (Bachteler et al., 2005; Bäckström et al., 2004; Vengeliene et al., 2007; Vengeliene et al., 2006).

### Effect of a H₃ Histaminergic Ligand Administration on Alcohol Relapse Behavior

The effects of a H₃ histaminergic ligand, such as L-histidine, are tested in our well-established model of relapse-like drinking behavior.

Alcohol Deprivation Effect (ADE) paradigm: All rats are given continuous access to tap water and to 5%, 10% and 20% (v/v) ethanol solutions in their home cages. Spillage and evaporation is minimized by the use of self-made bottles. All drinking solutions are renewed weekly and the positions of the four bottles are changed to avoid location preferences. After eight weeks of continuous access, ethanol solutions are repeatedly withdrawn for 3-21 days (deprivation phases) every four weeks until the rats become alcohol-dependent (as measured by a sucrose and quinine tests). The effects of a H₃ histaminergic ligand are then tested on the expression of the alcohol deprivation effect (=relapse behavior). A second ADE study is initiated 3 months later in order to study the persistence of treatment efficacy.

Past pharmacological studies in home cage drinking models were hampered by the fact that measurements were performed at 24 hour intervals. Thus the exact temporal profile of drug treatment could not be assessed under home cage drinking conditions and sometimes pharmacological effects became masked by the use of measurements every 24 hours. This problem has now been solved in our lab by the use of a fully-automated drinkometer system. In addition each animal is observed by an online e-motion tracking system that monitors home cage activity similar to a telemetric device, with the additional advantage that this is a complete non-invasive device. With this combined technology it is for the first time possible to monitor exactly micro-drinking patterns along with behavioral alterations.

The administration of L-histidine decreases relapse into alcohol drinking after alcohol deprivation (alcohol deprivation effect).

### Effect of H₃ Histaminergic Agonist Administration on Alcohol Craving Behavior

The goal of this study is to evaluate the effects of a H₃ histaminergic ligand, such as L-histidine, in a gold-standard model in the alcohol field. Namely, cue-induced reinstatement of alcohol-seeking behavior in order to measure alcohol-seeking responses (craving).

Priming and cue-induced reinstatement of ethanol-seeking behavior: Rats are trained to self-administer 10% (v/v) ethanol in a single 30 minute daily session on a Fixed Ratio 1 (FR 1) schedule. During the first three days of training, responses at the left lever are reinforced by delivery of 0.2% (w/v) saccharin solution. Following acquisition of saccharin-reinforced responding, rats are trained to self-administer ethanol. Thus, rats have access to 0.2% saccharin with 5% ethanol for one day, 5% ethanol for 1 day, 0.2% saccharin with 8% ethanol for 1 day, 8% ethanol for 1 day, 0.2% saccharin with 10% ethanol for 1 day, and 10% ethanol for 1 day. During all the training phase, responses at the right lever result in the delivery of a drop of water.

During the conditioning phase, animals are trained to discriminate the availability of ethanol (reward) vs. water (non-reward). This phase starts after completion of the saccharin-fading procedure. Discriminative stimuli predicting 10% ethanol or water availability are presented during each ethanol or water daily self-administration session (one 30 minutes session/day). An orange flavor extract serves as the S+ for ethanol, whereas water availability is signaled by anise extract (S−). These olfactory stimuli are generated by depositing six drops of the respective extract into the bedding of the operant chamber before the session, and remain present throughout the 30 minutes sessions. In addition, each lever press results in alcohol delivery accompanied by a 5 seconds auditory stimulus (“beep”, CS+), whereas a 5 seconds light stimulus (CS−) is presented with water delivery. The 5 second period serves as “time-out” during which responses are recorded but not reinforced. After completion of the conditioning phase, rats are subject to daily 30 minutes extinction sessions for 15 consecutive days which are usually sufficient to reach the extinction criterion of <10 lever responses/session. Extinction sessions begin by extension of the levers without presentation of the olfactory discriminative stimuli.

Reinstatement tests begin one day after the final extinction session. In these tests, rats are exposed to the same conditions as during the conditioning phase, except that liquids (alcohol or water) are not be available. Sessions are initiated by extension of both levers and presentation of either the alcohol-(S+) or water-(S−) associated discriminative stimuli. Responses at the each lever are followed by activation of the syringe pump motor and presentation of the CS+ (white noise) in the S+ condition or the CS− (house light) in the S− condition. Half of the animals are tested under the S+/CS+ condition on day 1 and under the S−/CS− condition on day 2. Conditions are reversed for the second half of the animals.

The number of responses on both the active lever (i.e., alcohol-associated lever for S+/CS+ condition and water-associated lever for S−/CS− condition) and the inactive lever (i.e., water-associated lever for S+/CS+ condition and alcohol-associated lever for S−/CS− condition) are recorded throughout the experiment. The effects of an acute injection of a H₃ histaminergic ligand are then tested on cue-induced reinstatement of ethanol-seeking behavior. In a further test, alcohol priming is tested as well as a combination of priming and cue-induced reinstatement of alcohol seeking behavior.

The administration of L-histidine decreases alcohol seeking (craving) induced by cues.

All publications such a textbooks, journal articles, Genbank or other sequence database entries, published applications and patent applications mentioned in this specification are herein incorporated by reference to the same extent as if each individual publication or patent was specifically and individually indicated to be incorporated by reference.

### REFERENCES

Ahmed, S. H., & Koob, G. F. Cocaine—but not food-seeking behavior is reinstated by stress after extinction. Psychopharmacology (Berl) 132:289-95 (1997).
Avena, N. M., et al. Evidence of sugar addiction: behavioral and neurochemical effects of intermittent, excessive sugar intake. Neuroscience & Behavioral Reviews 32:20-39 (2008).
Bachteler, D., et al. The effects of acamprosate and neramexane on cue-induced reinstatement of ethanol-seeking behaviour. Neuropsychopharmacology 30:1104-1 (2005).
Bäckström, S., et al. The mGluR5 antagonist MPEP prevents alcohol seeking and relapse behavior. Neuropsychopharmacology 29:921-928 (2004).
Block, W. D., et al. Histidine metabolism in the human adult: histidine blood tolerance, and the effect of continued free L-histidine ingestion on the concentration of imidazole compounds in blood and urine. J. Nutr. 91:189-94 (1967)
Boehm, S. L., II et al. Gamma-aminobutyric acid A receptor subunut mutant mice: new perspectives on alcohol actions. Biochem. Pharmacol. 68:1581-1602 (2004).
Borgland, S. L., et al. Acute and chronic cocaine-induced potentiation of synaptic strength in th ventral tegmental area: electrophysiological and behavioral correlates in individual rats. J. Neurosci. 24:7482-7490 (2004).
Bossong, M. G., et al. Delta 9-tetrahydrocannabinol induces dopamine release in the human striatum. Neuropsychopharmacology. 34:759-66 (2009).
Bostwick, J. M., & Bucci, J. A. Internet sex addiction treated with naltrexone. Mayo Clinic Proceedings 83:226-230 (2008).
Brabant, C., et al. Involvement of the brain histaminergic system in addiction and addiction-related behaviors: A comprehensive review with emphasis on the potential therapeutic use of histaminergic compounds in drug dependence. Progress in Neurobilogy 92:421-441 (2010).
Buczek, Y., et al., Stress reinstates nicotine seeking but not sucrose solution seeking in rats. Psychopharmacology (Berl) 144:183-8 (1999).
Caine, S. B., et al., Method for training operant responding and evaluating cocaine self-administration behavior in mutant mice. Psychopharmacology 147: 22-24 (1999).
Centers for Disease Control and Prevention. Annual Smoking—Attributable Mortality, Years of Potential Life Lost, and Productivity Losses—United States, 1997-2001. Morbidity and Mortality Weekly Report 54:625-628 (2005).
Childress, A. R., et al. Classically conditioned factors in drug dependence, 56-69 (In: Lowinson J, Ruiz P, Millman R, editors. Comprehensive Textbook of Substance Abuse. Williams & Wilkins; Baltimore, 1993).
Childress, A. R., et al. Limbic activation during cue-induced cocaine craving. Am. J. Psychiatry 156:11-8 (1999).
Dagher, A. & Robbins, T. W. Personality, addiction, dopamine: insights from Parkinson's disease. Neuron 61:502-510 (2009).
Dani, J. A., et al. Synaptic plasticity and nicotine addiction. Neuron 21:349-352 (2001).
de Wit, H. Priming Effects With Drugs and Other Reinforcers, Experimental and Clinical Psychopharmacology 4: 5-10 (1996).
Di Chiara, G., et al. Dopamine and drug addiction: the nucleus acumbens shell connection. Neuropharmacology 47(Suppl.):227-241 (2004).
Driver-Dunckley, E., et al. Gambling and increased sexual desire with dopaminergic medications in restless legs syndrome. Clinical Neuropharmacology 30:249-255 (2007).
Ehrman, R. N., et al. Responding to drug-related stimuli in humans as a function of drug-use history. NIDA Res. Monogr. 116:231-244 (1992).
Erb, S., et al. Stress reinstates cocaine-seeking behavior after prolonged extinction and a drug-free period. Psychopharmacology (Berl) 128:408-12 (1996).
Everitt, B. J. & Wolf, M. E. Psychomotor stimulant addiction: a neural systems perspective. J. Neurosci. 22:3312-3320 (2002).
Grant, J. E., et al. Introduction to behavioral addictions. Am. J. Drug Alcohol Abuse 36: 233-241 (2010).
Han, D. H. et al. Dopamine genes and reward dependence in adolescents with excessive internet video game play. Journal of Addiction Medicine 1:133-138 (2007).
Harwood, H. Updating Estimates of the Economic Costs of Alcohol Abuse in the United States: Estimates, Update Methods, and Data Report. Prepared by the Lewin Group for the National Institute on Alcohol Abuse and Alcoholism (2000).
Hill, S. J., et al. International Union of Pharmacology. XIII. Classification of histamine receptors. Pharmacol. Rev. 49:253-78 (1997).
Holier, S. M. & Spanagel, R. The effects of opiate antagonist treatment on the alcohol deprivation effect in long-term ethanol-experienced rats. Psychopharmacology 145:360-369 (1999).
Hölter, S. M., et al. Evidence for alcohol anti-craving properties of memantine. Eur. J. Pharmacol. 314:1-2 (1996).
Hölter, S. M., et al. Kappa-opioid receptors and relapse-like drinking in long-term ethanol-experienced rats. Psychopharmacology 153:93-102 (2000a).
Hölter, S. M., et al. The non-competitive NMDA receptor antagonist MRZ 2/579 suppresses the alcohol deprivation effect in long-term alcohol drinking rats and substitutes the alcohol cue in a discrimination task. J. Pharmacol. Exp. Ther. 246:1-8 (2000b).
Hölter, S. M., et al. Time course of acamprosate action on operant self-administration following ethanol deprivation. Alcohol Clin. Exp. Res. 21:862-869 (1997).
Howlett, A. C., et al. Cannabinoid physiology and pharmacology: 30 years of progress. Neuropharmacology 47(Suppl.):345-358 (2004).
Hyytia, P., et al. Histamine H3-receptor antagonist thioperamide potentiates behaviural effects of cocaine. European Journal of Pharmaceutical Sciences 19:S25 (Abstract) (2003).
Jaffe, J. J., et al. Cocaine-induced cocaine craving. Psychopharmacology (Berlin) 97:59-64 (1989).
Johnson, P. M. & Kenny, P. J. Dopamine D2 receptor in addiction-like reward dysfunction and compulsive eating in obese rats. Nature Neuroscience 13:635-641 (2010).
Kalivas, P. W. Glutamate systems in cocaine addiction. Curr. Opin. Pharmacol. 4:23-29 (2004).
Kauer, J. A. Learning mechanisms of addiction: synaptic plasticity in the ventral tegmental area as a result of exposure to drugs of abuse. Annu. Rev. Physiol. 66:447-475 (2004).
Kelley, A. B. & Berridge, K. C. The neuroscience of natural rewards: relevance to addictive drugs. J. Neurosci. 22:3306-3311 (2002).
Koob, G. F. & Le Moal, M. Drug addictions, dysregulation of rewards, and allostasis. Neuropsychopharmacology 24:97-129 (2001).
Lê, A. & Shaham, Y. Neurobiology of relapse to alcohol in rats. Phaimacol. Ther. 94:137-56 (2002).
Lê, A. D., et al. Reinstatement of alcohol-seeking by priming injections of alcohol and exposure to stress in rats. Psychopharmacology (Berl) 135:169-74 (1998).
Lintunen, M., et al. Increased brain histamine in an alcohol-preferring rat line, and modulation of ethanol comsumption by H3 receptor mechanisms. FASEB J. 15:1074-1076 (2001).
Lu, L., et al. Molecular neuroadaptations in the accumbens and ventral tegmental area during the first 90 days of forced abstinence from cocaine self-administration in rats. J. Neurochem. 85:1604-1613 (2003).
Munzar, P., et al. Histamine H3 receptor antagonists potentiate methamphetamine self-administration and metamphetamine-induced accumbal dopamine release. Neuropsychopharmacology 29:715-717 (2004).
Nestler, E. J. Molecular basis of long-term plasticity underlying addiction. Nat. Rev. Neurosci. 2:119-128 (2001).
Nestler, E. J. Molecular mechanisms of drug addiction. J. Neurosci. 12:2439-2450 (1992).
Nestler, E J. Is there a common molecular pathway for addiction. Nature Neuroscience 8:1445-1449 (2005).
Office of National Drug Control Policy. The Economic Costs of Drug Abuse in the United States: 1992-2002. Washington, D.C.: Executive Office of the President (Publication No. 207303) (2004).
Pidoplichko, V. I., et al. Nicotine activates and desensitizes midbrain dopamine neurons. Nature 390:401-4 (1997).
Reagan-Shaw, S., et al. Dose translation from animal to human studies revisited. FASEB J. 22:659-661 (2008).
Robinson, T. E. & Berridge, K. C. Addiction. Annu. Rev. Psychol. 54:25-53 (2003).
Rosell, S., et al. Automated HPLC method for determination of dopamine synthesis and release in brain samples. Manuscript in preparation.
Saal, D., et al. Drugs of abuse and stress trigger a common synaptic adaptation in dopamine neurons. Neuron 37:577-582 (2003).
Shaffer, J. P. Multiple Hypothesis Testing. Ann. Rev. Psych. 46, 561-584 (1995).
Soria, G., et al. Lack of CB1 cannabinoid receptor impairs cocaine self-administration. Neuropsychopharmacology 30:1670-1689 (2005).
Spanagel R. Alcohol addiction research: From animal models to clinics. Best Pract. Res. Cl. GA 17:507-518 (2003).
Spanagel R. Alcoholism: a systems approach from molecular physiology to addictive behavior. Physiol Rev. 89:649-705 (2009).
Spanagel, R. & Hölter, S. M. Long-term alcohol self-administration with repeated alcohol deprivation phases: An animal model of alcoholism? Alcohol and Alcoholism 34:231-243 (1999).
Spanagel, R. & Hölter, S. M. Pharmacological validation of a new animal model of alcoholism. J. Neural Transm. 107:669-80 (2000).
Spanagel, R. & Kiefer, F. Drugs for relapse prevention of alcoholism—10 years of progress. Trends Pharmacol. Sci. 29:109-115 (2008).
Spanagel, R. & Zieglgänsberger, W. Anti-craving compounds: new pharmacological tools to study addictive processes. Trends Pharmacol. Sci. 18:54-9 (1997).
Spanagel, R. Alcoholism—a systems approach from molecular physiology to behavior. Physiol. Rev. 89:649-705 (2009).

Spanagel, R., et al. Acamprosate and alcohol: I. Effects on alcohol intake following alcohol deprivation in the rat. Eur. J. Phaimacol. 305:39-44 (1996).
Tamming a, C. A. & Nestler, E. J. Pathological Gambling: focusing on the addictions, not the activity. Am. J. Psychiatry 163:180-181 (2006).
Thomas, M. J. & Malenka, R. C. Synaptic plasticity in the mesolimbic popamine system. Phil. Trans. R. Soc. Lond. B Biol. Sci. 358:815-819 (2003).
Tobler, P. N., et al. Adaptive coding of reward value by dopamine neurons. Science 307:1642-1645 (2005).
Vengeliene, V., et al. The D3 receptor plays an essential role in alcohol craving and relapse but not in alcohol reinforcement processes. FASEB J. 20:2223-2233 (2006).
Vengeliene, V., et al. The effect of lamotrigine on alcohol seeking and relapse. Neuropharmacology 53:951-795 (2007).
Vengeliene, V., et al. The role of the NMDA receptor complex in alcohol relapse: A pharmacological mapping study using the alcohol deprivation effect. Neuropharmacology 48: 822-829 (2005).
Verheul, R., et al. A three-pathway psychobiological model of craving for alcohol. Alcohol Alcoholism 34:197-222 (1999).
Volkow, N. D., et al. Dopamine in drug abuse and addiction: results from imaging studies and treatment implications. Mol. Psychiatry. 9:557-569 (2004).
Voon, V. & Fox, S. H. Medication-related impulse control and repetitive behaviours in Parkinson disease. Arch. Neurol. 20:484-492 (2007).
Voon, V., et al. Medication-related impulse control and repetitive behaviors in Parkinson's disease. Curr. Opin. Neurol. 20:484-492 (2007).
Wallace, B. Psychological and environmental determinants of relapse in crack cocaine smokers. J. Subst. Abuse Treat. 6:95-106 (1989).
Weintraub, D., et al. Association of dopamine agonist use with impulse control disorders in Parkinson disease. Arch. Neurol. 63, 969-973 (2006).
Wise, R. A. Dopamine, learning and motivation. Nat. Rev. Neurosci. 5: 483-494 (2004).
Wu, X., et al. Carnosine, a precursor of histidine, ameliorates pentylenetetrazole-induced kindled seizures in rat. Neuroscience Letters 400:146-149 (2006).
Zolman, N. Biostatistics: Experimental Design and Statistical Inference. New York: Oxford University Press (1993).

What is claimed is:

1. A method for altering addiction-related behavior in a subject suffering from addiction comprising,
administering to said subject a therapeutically effective amount of an H₃ histaminergic agonist,
wherein said effective amount of an H₃ histaminergic agonist is sufficient to diminish, inhibit or eliminate the addiction-related behavior.

2. A method for altering addiction-related behavior in a subject suffering from addiction to at least one drug of abuse comprising,
administering to said subject a therapeutically effective amount of an H₃ histaminergic agonist,
wherein said effective amount of an H₃ histaminergic agonist is sufficient to diminish, inhibit or eliminate the addiction-related behavior.

3. The method of claim 2, wherein said drug of abuse causes an increase in dopaminergic transmission in the nucleus accumbens area of the human brain by direct interaction with neurons located in the nucleus accumbens.

4. The method of claim 2, wherein said drug of abuse causes an increase in dopaminergic transmission in the nucleus accumbems area of the human brain by interacting with neurons located in other areas of the central nervous system.

5. The method of claim 4, wherein said drug of abuse activates or inhibits neuronal transmission in the ventral tegmental area of the human midbrain.

6. The method of claims 1-5, wherein said addiction-related behavior is craving.

7. The method of claims 1-6, wherein said addiction-related behavior is relapse.

8. A method for treating or ameliorating the symptoms of addiction to drugs of abuse comprising,
administering to a subject in need thereof a therapeutically effective amount of an H₃ histaminergic agonist,
wherein said effective amount promotes extinction.

9. A method for treating or ameliorating the symptoms of addiction to drugs of abuse comprising
administering to a subject in need thereof a therapeutically effective amount of an H₃ histaminergic agonist,
wherein said effective amount inhibits relapse.

10. A method for treating or ameliorating the symptoms of addiction to drugs of abuse comprising
administering to a subject in need thereof a therapeutically effective amount of an H₃ histaminergic agonist,
wherein said effective amount inhibits self-administration.

11. The method of any of claims 1-10, wherein said H₃ histaminergic agonist is selected from the group consisting of imetit, immepip, and methyl histamine.

12. The method of any of claims 1-10, wherein said H₃ histaminergic agonist is an agonist precursor.

13. The method of claim 12, wherein said agonist precursor is L-histidine.

14. The method of claim 12, wherein said agonist precursor is a prodrug.

15. The method of any of claims 1-14, wherein said drug of abuse is selected from the group consisting of opiates, hallucinogens, inhalants, phencyclidine, amphetamines, cocaine, cannabis, nicotine, and alcohol.

16. The method of any of claims 1-14 wherein said drug of abuse is selected from the group consisting of cocaine, ethanol, and nicotine.

17. The method of any of claims 1-14, wherein said drug of abuse is a combination of drugs.

18. The method of any of claims 1-17, wherein said treatment further comprises administering an additional therapeutic agent.

19. The method of claim 18, wherein the additional therapeutic agent is administered concurrently.

20. The method of claim 18, wherein the additional therapeutic agent is administered sequentially.
